(19) United States
(12) Patent Application Publication
(10) Pub. No.: US 2011/0093364 A1
(43) Pub. Date: Apr. 21, 2011

(54) SYSTEMS, METHODS AND COMPUTER PRODUCTS FOR PHARMACEUTICAL SAMPLES MANAGEMENT

(76) Inventor: KAMAL SHARMA, Parsippany, NJ (US)

(21) Appl. No.: 12/759,674

(22) Filed: Apr. 13, 2010

Related U.S. Application Data
(60) Provisional application No. 61/175,440, filed on May 4, 2009.

(51) Int. Cl. G06Q 10/00; G06F 17/30

(52) U.S. Cl. 705/28; 707/602; 707/769; 707/E17.009; 707/E17.014

(57) ABSTRACT

A system for pharmaceutical samples management is provided, the system including: (1) an allocation module adapted to allow a user to create allocation models for pharmaceutical samples, wherein an allocation model specifies quantities of pharmaceutical samples that are allocated amongst a sales team for specified time periods; (2) an order module adapted to allow the user to order pharmaceutical samples based on an allocation model; (3) a transaction module adapted to allow the user to monitor a transaction in the system; (4) a reconciliation module adapted to allow the user to perform samples inventory reconciliation; (5) a reports module adapted to allow the user to obtain reports regarding the system; (6) an analytics module adapted to allow the user to obtain analytical information regarding the system; (7) a dashboard module adapted to allow the user to display data regarding the system; and (8) an alerts module adapted to provide alert information regarding the system. Numerous other aspects are provided.

### REFERENCE TO RELATED APPLICATIONS

This application claims the benefit of U.S. provisional patent application Ser. No. 61/175,440, filed on May 4, 2009, and titled “Systems And Methods For Pharmaceutical Samples Management,” which is incorporated by reference herein in its entirety for all purposes.

### TECHNICAL FIELD

This invention relates to systems and methods for pharmaceutical samples management.

### BACKGROUND

Pharmaceutical companies routinely distribute free samples of their prescription drugs to prescribers, such as doctors and nurse practitioners. Such samples programs permit doctors to titrate treatment programs at no cost to their patients, and also enable pharmaceutical companies to build brand awareness and visibility in target markets. Because samples are provided at no cost to the prescribers or patients, pharmaceutical companies bear the entire cost for manufacturing, distributing, tracking, and managing the samples. The cost of such samples management is substantial. Indeed, the cost of samples production and management is typically one of the highest costs in a brand's sales and marketing budget. Recent estimates of the industry-wide cost of manufacturing and managing samples is about $18 billion. Accordingly, pharmaceutical companies seek ways to increase their return on investment for samples programs.

Because of the substantial costs associated with samples manufacturing, distribution and management, improved tools for reducing costs and increasing efficiency in pharmaceutical samples programs are desirable.

### SUMMARY

In a first aspect of the invention, a system for pharmaceutical samples management is provided, the system including: (1) an allocation module adapted to allow a user to create allocation models for pharmaceutical samples, wherein an allocation model specifies quantities of pharmaceutical samples that are allocated amongst a sales team for specified time periods; (2) an order module adapted to allow the user to order pharmaceutical samples based on an allocation model; (3) a transaction module adapted to allow the user to monitor a transaction in the system; (4) a reconciliation module adapted to allow the user to perform samples inventory reconciliation; (5) a reports module adapted to allow the user to obtain reports regarding the system; (6) an analytics module adapted to allow the user to obtain analytical information regarding the system; (7) a dashboard module adapted to allow the user to display data regarding the system; and (8) an alerts module adapted to provide alert information regarding the system.

In a second aspect of the invention, a system for pharmaceutical samples management, the system including: (1) means for creating allocation models that specify quantities of samples that are allocated for specified time periods; (2) means for attaching schedules to the allocation models to specify time periods for providing feedback, ordering samples and receiving shipments of samples; (3) means for providing feedback regarding quantities specified in allocation models; (4) means for ordering samples allocated in allocation models; (5) means for tracking shipments of ordered samples; (6) means for providing inventory data regarding samples; and (7) means for analyzing data regarding samples programs.

In a second aspect of the invention, a computer program product for implementing a pharmaceutical samples management system is provided, the computer program product including a machine readable storage medium, machine code stored in the machine readable storage medium which when executed by a computer processor configures the computer processor to: (1) allow a user to create allocation models for pharmaceutical samples, wherein an allocation model specifies quantities of pharmaceutical samples that are allocated amongst a sales team for specified time periods; (2) allow the user to order pharmaceutical samples based on an allocation model; (3) allow the user to monitor a system transaction; (4) allow the user to perform samples inventory reconciliation; (5) allow the user to obtain reports regarding the system; (6) allow the user to obtain analytical information regarding the system; (7) allow the user to display data regarding the system; and (8) provide alert information regarding the system.

Other features and aspects of the present invention will become more fully apparent from the following detailed description, the appended claims and the accompanying drawings.

### BRIEF DESCRIPTION OF THE DRAWINGS

Features of the present invention can be more clearly understood from the following detailed description considered in conjunction with the following drawings, in which the same reference numerals denote the same elements throughout, and in which:

FIG. 1 is a block diagram of an exemplary system in which a pharmaceutical samples management system in accordance with this invention may be used;

FIG. 2 is a block diagram of an exemplary pharmaceutical samples management system in accordance with this invention;

FIG. 3 is a diagram of an exemplary database in accordance with this invention;

FIG. 4 is a diagram of an exemplary schedule used by systems in accordance with this invention;

FIG. 5 is a block diagram of an exemplary Allocation Module in accordance with this invention;

FIG. 6 is a block diagram of an exemplary Allocation Model Creation Module in accordance with this invention;

FIG. 7 is a block diagram of an exemplary Order Module in accordance with this invention;

FIG. 8 is a block diagram of an exemplary Order Creation Module in accordance with this invention;

FIG. 9 is a block diagram of an exemplary Transaction Module in accordance with this invention;

FIG. 10 is an exemplary system for providing pharmaceutical sample management in accordance with this invention;

FIG. 11 is a diagram of an exemplary administration user interface in accordance with this invention;

FIGS. 12A-12D are diagrams of exemplary allocation user interfaces in accordance with this invention;

FIGS. 13A-13C are diagrams of exemplary orders user interfaces in accordance with this invention;

FIGS. 14A-14B are diagrams of exemplary transaction manager user interfaces in accordance with this invention;

FIGS. 15A-15B are diagrams of exemplary reconciliation manager user interfaces in accordance with this invention;

FIGS. 16A-16B are diagrams of exemplary reports user interfaces in accordance with this invention;

FIG. 17 is a diagram of an exemplary dashboard user interface in accordance with this invention; and

FIG. 18 is a diagram of an exemplary notification-alerts user interface in accordance with this invention.

### DETAILED DESCRIPTION

As previously stated, costs associated with pharmaceutical samples programs are substantial. In addition to the costs to manufacture the actual medications, pharmaceutical companies incur substantial costs to distribute and monitor pharmaceutical samples. In particular, the Prescription Drug Marketing Act of 1987 (“PDMA”) mandates strict guidelines for the control, disbursement, shipment, and handling of all prescription drugs, and specifically imposes strict guidelines for the control and handling of samples. As a result, pharmaceutical companies seek ways to reduce costs, improve efficiency and scrupulously adhere with PDMA compliance requirements for sample programs.

Pharmaceutical companies typically have multiple organizational groups that collectively implement a pharmaceutical samples program. For example, a pharmaceutical company may include a “Samples Operations” group that manages the logistical and regulatory aspects of a samples program, a “Brand Team” that focuses on financial and budgeting aspects of the program, a “Warehouse” that controls manufacture and delivery of samples, and a “Sales Team” including sales representatives who receive samples from the Warehouse and then distribute the samples to prescribers.

Because of the number of organizations involved in samples programs, the complexities associated with manufacturing and distributing medications that have a limited useful life to a large number of sales representatives, and the strict regulatory requirements for samples, pharmaceutical samples programs are quite demanding. Some previously known software systems have been developed to facilitate implementation of pharmaceutical samples programs. However, existing systems typically focus on PDMA compliance. For example, available software applications typically provide a front-office interface to capture and track disbursement, delivery and receipt of samples from the warehouse to pharmaceutical sales representatives, and/or a back-office interface used by corporate users for samples tracking and reconciliation.

Although existing samples management systems provide some useful functionality, the systems fall far short of providing solutions that could enable pharmaceutical companies to improve the efficiency and reduce the cost of their samples programs. In particular, areas for cost reduction include (1) improved allocation planning and management, (2) reduction of waste (e.g., expiring product that must be returned for destruction), (3) prevention of excess samples inventory, (4) reduction of administrative costs and (5) improvement of samples reconciliation.

Aside from the limited front-office/back-office features described above, existing samples management software do not provide comprehensive integrated solutions that allow pharmaceutical companies to implement samples programs, and track, measure and control their costs with respect to such programs. Indeed, no existing samples management system integrates data for samples tracking and reporting, business planning, demand forecasting, event-based alerting, and data extraction and analysis so that the information can be used to optimize and improve efficiency of samples programs.

Instead, pharmaceutical companies today typically rely on a variety of different software applications that individually focus on specific sub-tasks of an entire samples management process. For example, the Sales Team may use a sales force automation or customer relations management software program that is used for other sales activities to also acknowledge receipt of samples from the Warehouse, report movement or loss of samples, and report on current samples inventory. At the same time, Samples Operations and the Brand Team may utilize a different set of software applications for managing samples oversight, inventory reconciliation, and samples management tasks. This fragmentation of samples management tasks amongst a variety of task-specific software applications limits the ability of pharmaceutical companies to implement comprehensive and cross-functional process control and measurement solutions that can reduce costs and improve the efficiency of samples management programs. Further, the use of such fragmented solutions increases the cost and complexity of samples management programs, and makes it difficult to quickly monitor and adapt business processes based on dynamic market conditions.

Methods and system accordance with this invention provide integrated tools that may be used to manage, analyze data, report, and communicate information for pharmaceutical samples programs based on data in samples management databases. Systems in accordance with this invention may be implemented as a web-based solution, a server-based application, or any other similar implementation.

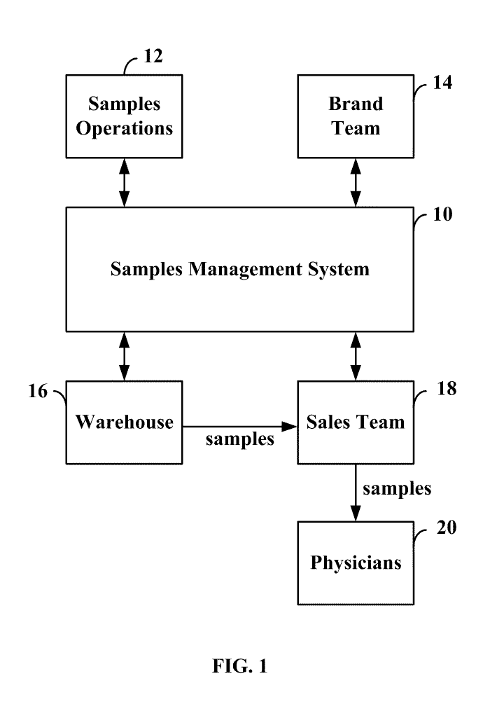

Referring now to FIG. 1, an exemplary pharmaceutical samples management system 10 in accordance with this invention is described. In particular, samples management system 10 is illustrated as implemented in an organization that includes various functional groups, such as Samples Operations 12, Brand Team 14, Warehouse 16 and Sales Team 18. Persons of ordinary skill in the art will understand that samples management systems in accordance with this invention may be used with more, fewer, or different functional groups than the exemplary groups shown in FIG. 1. Indeed, in accordance with this invention, samples management system 10 includes tools that may be flexibly used by a variety of different organizational structures.

In accordance with this invention, samples management system 10 enables Samples Operations 12, Brand Team 14, Warehouse 16 and Sales Team 18 (collectively referred to as “Users”) to design and implement programs for distributing samples to Prescribers 20, and to track, monitor and control costs with respect to such programs. In particular, samples management system 10 enables Users to prepare budgets, place orders, monitor compliance, perform recordkeeping, analyze performance and display data regarding samples programs.

Even more particularly, samples management system 10 provides an integrated solution that enables Users to: (1) create allocation models that specify quantities of samples that are allocated amongst Sales Team 18 for specified time periods; (2) attach schedules to the allocation models to specify time periods for providing feedback, ordering samples and receiving shipments of samples; (3) provide feedback regarding quantities specified in allocation models; (4) order samples allocated in allocation models; (5) track shipments of ordered samples; (6) provide inventory data regarding samples; and (7) analyze data regarding samples programs.

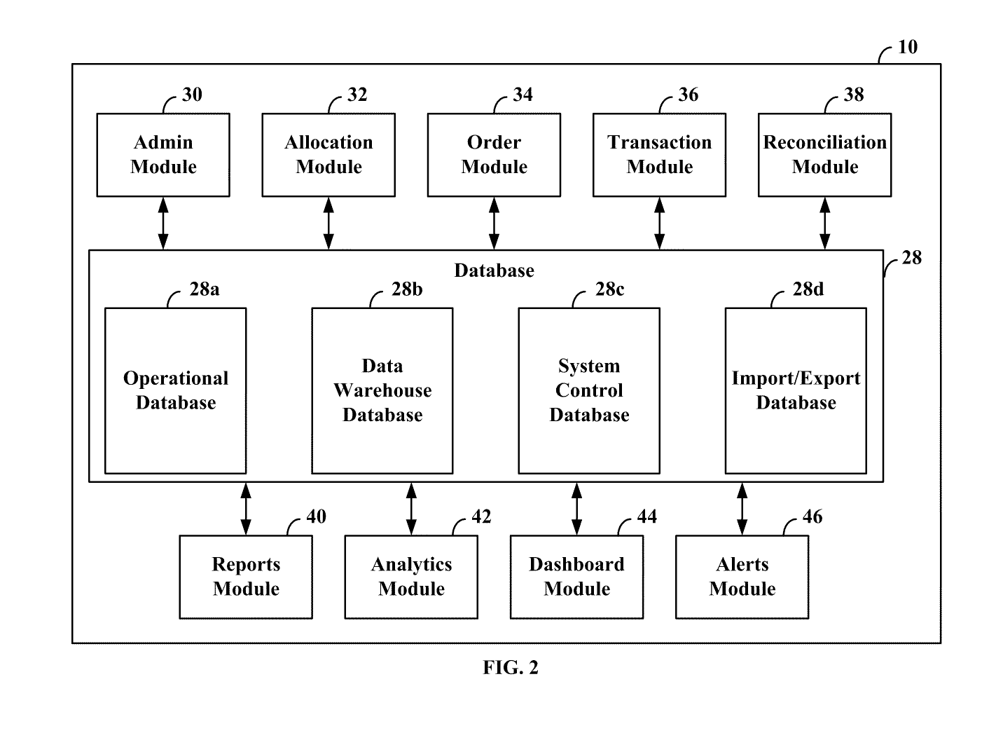

An exemplary samples management system 10 is illustrated in FIG. 2, and includes Database 28, Administration Module 30, Allocation Module 32, Order Module 34, Transaction Module 36, Reconciliation Module 38, Reports Module 40, Analytics Module 42, Dashboard Module 44, and Alerts Module 46. Although not shown in FIG. 2, samples management system 10 also includes a user interface that enables Users to interact with Modules 30-46 to design, implement and monitor samples programs. Persons of ordinary skill in the art will understand that systems in accordance with this invention may include more, fewer and/or different modules than the ones illustrated in FIG. 2.

Database 28 and Modules 30-46 and may be co-located at a single location, or may be distributed amongst multiple locations. Database 28 may include a single database, or a collection of multiple databases. In the exemplary embodiment of FIG. 2, Database 28 includes Operational Database 28a, Data Warehouse Database 28b, System Control Database 28c, and Import/Export Database 28d. Persons of ordinary skill in the art will understand that Database 28 may include more, fewer and/or different databases than the ones illustrated in FIG. 2.

Operational Database 28a stores data in a form suitable for transaction processing, and may include one or more sub-databases, described in more detail below. Data Warehouse Database 28b stores data extracted from Operational Database 28a in a form suitable for use by Reports Module 40 and Analytics Module 42, described below. System Control Database 28c contains system configuration elements, such as database connectivity, system folder structures and files names, web server configuration details and other similar system details. Import/Export Database 28d stores data imported from and/or exported to external sources and samples management system 10.

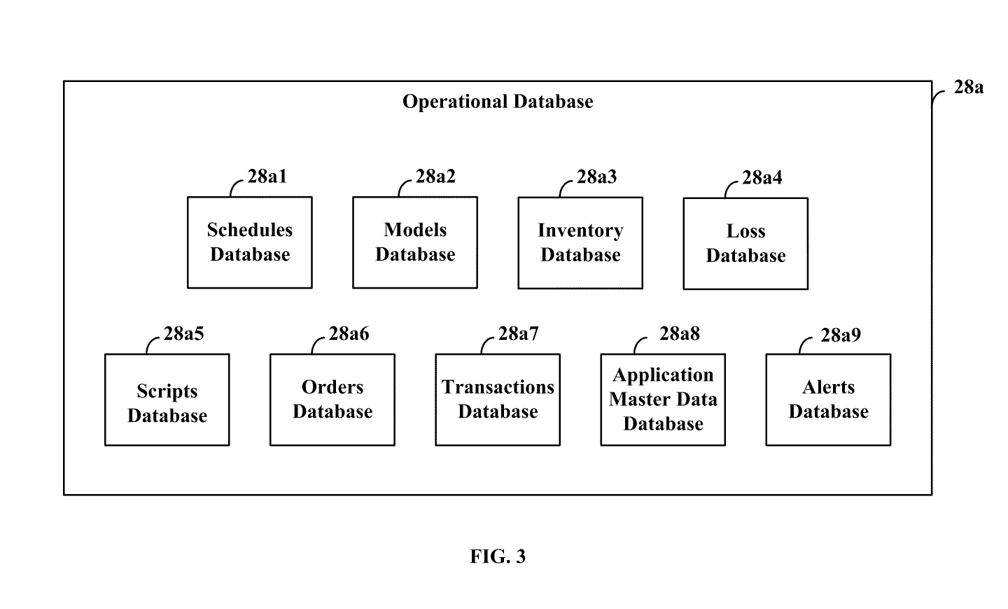

Referring now to FIG. 3, an exemplary Operational Database 28a is now described. Operational Database 28a includes Schedules Database 28a1, Models Database 28a2, Inventory Database 28a3, Loss Database 28a4, Scripts Database 28a5, Orders Database 28a6, Transactions Database 28a7, Application Master Data Database 28a8, and Alerts Database 28a9. Persons of ordinary skill in the art will understand that Operational Database 28a may include more, fewer, or different databases than exemplary databases 28a1-28a9 illustrated in FIG. 3. Each of databases 28a1-28a9 will be discussed in turn.

Schedules Database 28a1 stores data regarding various schedules used by samples management system 10. In accordance with an exemplary embodiment of this invention, samples management system 10 uses the following schedules, which are stored in Schedules Database 28a1: an Allocation Feedback Schedule, an Orders Schedule, a Shipping Schedule and an Inventory Schedule. Each of these schedules will be described in more detail below. Persons of ordinary skill in the art will understand that samples management system 10 may use more, fewer or different schedules than the four exemplary schedules described. A system administrator (not shown) may create and/or modify the schedules using Administration Module 30.

Models Database 28a2 stores allocation models created or imported by Users using Allocation Module 32. As commonly used in the pharmaceutical industry, an “Allocation” specifies quantities of one or more samples that are budgeted for a specific time period, and further specifies how the quantities are subdivided into specific quantities of samples allocated amongst the Sales Team for the specified time period. Allocations are typically subdivided into the most granular Sales Team entity level, which is generally the Territory level. Thus, an allocation will typically specify the quantities of samples that will be allocated to each individual Territory for a specific time period.

Inventory Database 28a3, Loss Database 28a4, and Scripts Database 28a5 store data regarding existing samples inventory on hand, prior samples loss rates, and prior quantities of prescriptions written for each sample, respectively, for each Territory. Orders Database 28a6 stores order data created or imported by Users using Order Module 34.

Transactions Database 28a7 stores “Transaction Records” of activities that occur in samples management system 10. Exemplary Transaction Records may include disbursements (e.g., deliveries of samples to Prescribers 20), shipments (e.g., shipments of samples from Warehouse 16 to Territories), inventory records, returns (e.g., expiring/damaged samples that must be returned), theft/loss, transfer-in and transfer-out records (e.g., transfers between Territories), adjustment records, and other similar records.

Application Master Data Database 28a8 contains “master data,” such as employee information, Sales Territory information, samples information, user administration information, and other similar data. Information in Application Master Data Database 28a8 may be edited in Command Module 30 by authorized Users.

Alerts Database 28a9 contains alerts generated by Alerts Module 46. This database also may store information about which alerts have been archived, are in-progress or new for each User.

Referring again to FIG. 2, and as previously mentioned, modules 30-46 allow Users to design, implement and monitor samples programs. Each of modules 30-46 will be described in turn.

### Administration Module

A system administrator may use Administration Module 28 to specify one or more schedules, such as Allocation Schedules, Order Schedules, Shipping Schedules and Inventory Schedules, defined as follows:
Allocation Schedule—specifies one or more Allocation Window time periods during which Users specified in an Allocation User List may use Allocation Module 30 to provide feedback regarding allocation models. Users not specified in the Allocation User List may not provide feedback during the active Allocation Window.
Order Schedule—specifies one or more Order Window time periods during which Users specified in an Order User List may access Order Module 34 to place orders for Samples based on corresponding allocation models. Users not specified in the Order User List may not place orders during the active Order Window.
Shipping Schedule—specifies one or more Shipping Window time periods during which Warehouse 16 will ship samples to Sales Team 18.
Inventory Schedule—specifies one or more Inventory Window time periods during which Sales Team 18 are required to report samples inventory. For example, Sales Team 18 Users may use a third party application, such as Microsoft Excel, to create an inventory list in an Excel spreadsheet. Administration Module 28 may then import the Excel spreadsheet and extract inventory data. Persons of ordinary skill in the art will understand that other similar third party applications may be used to provide inventory data.

In exemplary embodiments of this invention, a samples process cycle includes an Allocation Window, an Order Window and a Shipping Window. The Allocation Window typically will lead the entire process cycle. Once an Allocation Window closes, an Order Window typically opens. Once the Order Window closes, a Shipping Window typically opens. Once a Shipping Window closes, a cycle of the samples process completes. The next cycle begins with the opening of an Allocation Window. In exemplary embodiments of this invention, there is generally no interdependence between the Inventory Window and the Allocation Window, Order Window, or Shipping Window. In addition, in exemplary embodiments of this invention, a samples process cycle may be subdivided into one or more sub-cycles.

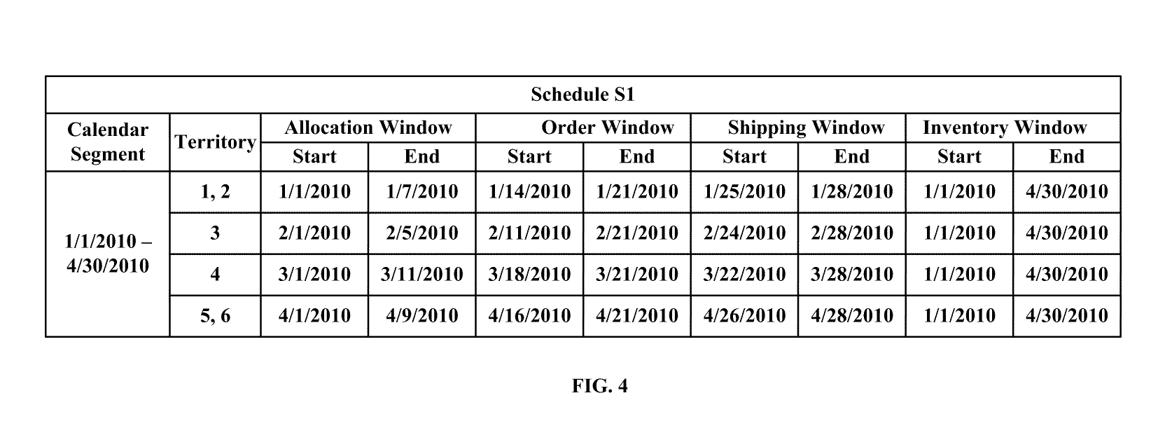

For example, FIG. 4 illustrates an exemplary schedule S1 for Territories 1-6, covering the time period Jan. 1, 2010 through Apr. 30, 2010. In particular, schedule S1 specifies that Territories 1 and 2 have an Allocation Window that opens on Jan. 1, 2010 and closes on Jan. 7, 2010, an Order Window that opens on Jan. 14, 2010 and closes on Jan. 21, 2010, and a Shipping Window that opens on Jan. 25, 2010 and closes on Jan. 28, 2010. During the open Allocation Window, only Territories 1 and 2 may access Allocation Module 30 to provide feedback regarding allocation models attached to schedule S1. Likewise, during the open Order Window, only Territories 1 and 2 may access Order Module 34 to place orders for samples based on corresponding allocation models attached to schedule S1. Samples orders placed during the Jan. 14, 2010 through Jan. 21, 2010 Order Window are scheduled for shipment during the Jan. 25, 2010 through Jan. 28, 2010 Shipping Window.

In contrast, schedule S1 specifies that Territory 4 has an Allocation Window that opens on Mar. 1, 2010 and closes on Mar. 11, 2010, an Order Window that opens on Mar. 18, 2010 and closes on Mar. 21, 2010, and a Shipping Window that opens on Mar. 22, 2010 and closes on Mar. 28, 2010. During the open Allocation Window, only Territory 4 may access Allocation Module 30 to provide feedback regarding allocation models attached to schedule S1. Likewise, during the open Order Window, only Territory 4 may access Order Module 34 to place orders for samples based on corresponding allocation models attached to schedule S1. Samples orders placed during the Mar. 18, 2010 through Mar. 21, 2010 Order Window are scheduled for shipment during the Mar. 22, 2010 through Mar. 28, 2010 Shipping Window. Moreover, during the entire schedule, an Inventory Window remains open for Territories 1-6.

The time periods and durations specified above are for illustrative purposes only. Persons of ordinary skill in the art will understand that the duration of the various Allocation Windows, Order Windows and Shipping Windows may vary from Territory to Territory. In addition, although the exemplary Inventory Schedules illustrated in FIG. 4 span the entire calendar segment, persons of ordinary skill in the art will understand that Inventory Schedules may be specified for shorter time periods.

### Allocation Module

Referring again to FIG. 2, Allocation Module 30 may be used to create allocation models, and to display, provide feedback, edit and approve quantities of samples specified in allocation models. As described above, an allocation specifies quantities of one or more samples that are budgeted for a specific time period, and further specifies the quantities of samples that will be allocated to each individual Territory for a specific time period. As described in more detail below, the allocated quantity of each sample sets a limit on the quantity of that sample that a Territory may order.

In accordance with this invention, Allocation Module 30 may be used to generate one or more allocation models, with each allocation model specifying an Allocation of samples amongst one or more Sales Territories for a particular time interval. Each allocation model may have an associated identifying model name (e.g., similar to a file name). This allows Users to create, save, and identify multiple allocation models, and easily distinguish between various allocation models.

For example, Table 1 indicates an exemplary allocation model “T12010” that specifies an Allocation of Medicines A, B and C to Territories 1, 2 and 3.

TABLE 1
Allocation Model Name: T12010

| Territory | Product | Quantity |
|---|---|---|
| 1 | Medicine A | 10 |
| 1 | Medicine B | 20 |
| 1 | Medicine C | 0 |
| 2 | Medicine A | 50 |
| 2 | Medicine B | 100 |
| 2 | Medicine C | 25 |
| 3 | Medicine A | 0 |
| 3 | Medicine B | 35 |
| 3 | Medicine C | 10 |

Thus, in this example, Territory 1 has been allocated 10 units of Medicine A, 20 units of Medicine B and no units of Medicine C, Territory 2 has been allocated 50 units of Medicine A, 100 units of Medicine B and 25 units of Medicine C, and Territory 3 has been allocated no units of Medicine A, 35 units of Medicine B and 10 units of Medicine C.

Allocation models, such as the exemplary allocation model in Table 1, may be used for a variety of purposes. Samples Operations 12, Brand Team 14, Warehouse 16 and Sales Team 18 may use an allocation model for logistical, financial production planning and sales planning purposes, respectively. For example, Brand Team 14 may use Allocation Module 30 to create a proposed allocation model, such as model T12010 shown in Table 1. Samples Operations 12 may then attach a schedule from Schedules Database 28a1 to allocation model T12010. For example, a Samples Operations 12 may attach schedule S1 (FIG. 4) to allocation model T12010. During the Allocation Windows specified in schedule S1, Sales Team 18 may provide feedback regarding samples quantities specified in allocation model T12010.

Thus, between Jan. 1, 2010 and Jan. 7, 2010, Territory 1 and 2 may use Allocation Module 30 to provide feedback regarding samples quantities specified for Territories 1 and 2 in allocation model T12010. Likewise, between Feb. 1, 2010 and Feb. 5, 2010, Territory 3 may use Allocation Module 30 to provide feedback regarding samples quantities specified for Territory 3 in allocation model T12010. After the close of each allocation window, Samples Operations 12 may use Allocation Module 30 to review the feedback, and may modify samples quantities specified in allocation model T12010. Samples Operations 12 may then approve quantities for each sample specified in allocation model T12010, and may then mark the model as “Reviewed.” The reviewed allocation model is then saved in Transactions Database 28a7.

Sales Team 18 may then order samples against the allocated quantities during the Order Window specified in schedule S1. For example, between Jan. 14, 2010 and Jan. 21, 2010, Territories 1 and 2 may order samples of medicines A, B and C based on the quantities specified in the reviewed allocation model T12010. In contrast, Territory 3 may only order samples of medicines A, B and C between Feb. 11, 2010 through Feb. 21, 2010. Any samples ordered during the specified Order Windows are shipped to Territories 1 and 2 between Jan. 25, 2010 and Jan. 28, 2010, and to Territory 3 between Feb. 24, 2010 and Feb. 28, 2010.

Although this example has described Brand Team 14 creating the allocation model, Samples Operations 12 attaching the schedule and reviewing the allocation model, and Sales Team 18 providing feedback about the allocation model, persons of ordinary skill in the art will understand that samples management systems in accordance with this invention are not limited to such a specific use. For example, Sales Team 18 may create a proposed allocation model, Brand Team 14 may provide feedback regarding the model, and Samples Operations 12 and Sales Team 18 may collectively review and approve the final quantities.

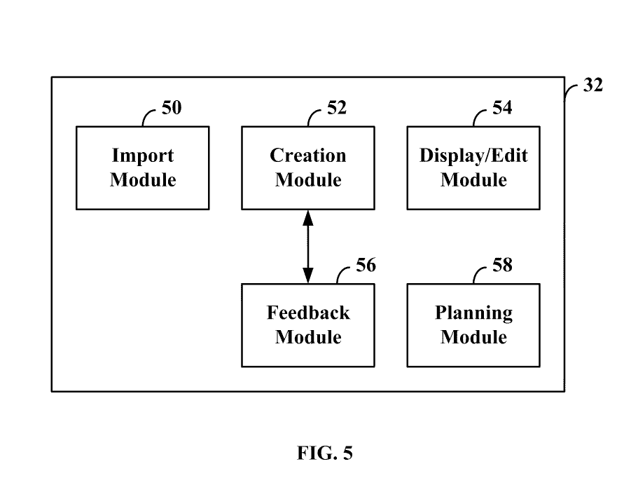

Referring now to FIG. 5, an exemplary Allocation Module 30 is described. Allocation Module 30 includes Import Module 50, Creation Module 52, Display/Edit Module 54, Feedback Module 56 and Planning Module 58. Each of the exemplary modules will be described in turn.

Import Module 50 enables a User to import “externally-generated” allocation models. For example, a User may create an allocation model using a spreadsheet program that is separate from Samples Management System 10. Import Module 50 enables the User to import the externally generated allocation model into Samples Management System 10 (e.g., via Import/Export Database 28d (FIG. 2)), and then store the imported allocation model in Models Database 28a2 (FIG. 3).

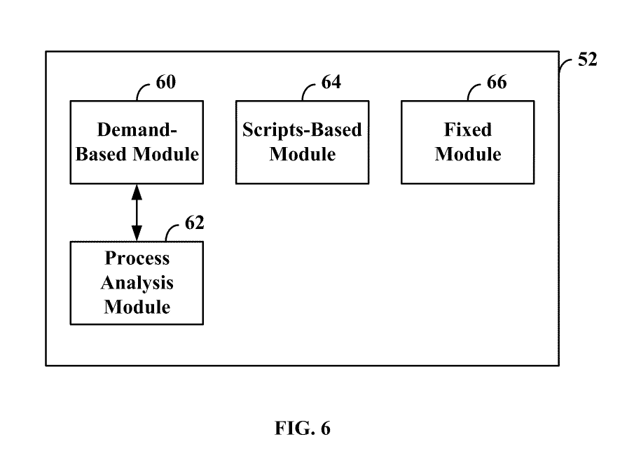

Creation Module 52 enables Users to create an allocation model within Samples Management System 10 and save the created allocation model to Models Database 28a2. For example, as illustrated in FIG. 6, Creation Module 52 may include Demand-Based Module 60, Process Analysis Module 62, Scripts-Based Module 64 and Fixed Module 66, each of which will be described in more detail below. Persons of ordinary skill in the art will understand that Creation Module 52 may include more, fewer or different modules than the ones illustrated in FIG. 6 for creating allocation models.

Demand-Based Module 60 may create allocation models based on historical samples data for each Territory. Referring again to FIG. 3, Inventory Database 28a3 may include data regarding the existing inventory on hand, and Loss Database 28a4 may include data regarding prior loss rates. Each of Databases 28a4-28a5 may include such data for each sample for each Territory. In accordance with this invention, Demand-Based Module 60 may create allocation models based on data in Inventory Database 28a3 and/or Loss Database 28a4.

In particular, a User may specify a desired time period, desired samples and desired Territories, and Demand-Based Module 60 may calculate quantities of the specified samples for the specified Territories based on prior usage data, existing inventory data and/or loss data, and may then create an allocation model for the specified time period for the specified Territories. For example, an allocation model for a particular Territory T₁, for a particular sample Medicine A for N weeks may be calculated as:

Sample Allocation=((B*N)+C+D)−A

where:

| | |
|---|---|
| A | The estimated quantity of Medicine A that Territory T₁ currently possesses. For |
| | example, the value A may be calculated as Territory T₁'s Reported Inventory + |
| | shipments after inventory + Transfer-In after inventory − Disbursements after |
| | inventory − Transfer-Out after inventory − Returns after inventory − Theft/Loss |
| | after inventory. |
| B | Actual disbursements of Medicine A by Territory T₁. For example, the value B |
| | may be calculated as the total quantity of Medicine A disbursed by Territory T₁ |
| | over X weeks)/X weeks. |
| C | All other non-disbursement transactions. For example, the value C may be |
| | calculated as the average amount of Medicine A that Territory T₁ (Transferred In − |
| | Transferred Out − Returned) in the past X weeks. |
| D | A buffer quantity of Medicine A to be included - for example in case shipments |
| | are delayed. |
| N | The number of weeks until next scheduled shipment. For example, the value N |
| | may be calculated as the number of weeks from current day until Territory T₁ is to |
| | receive the next shipment after delivery of the most current shipment. |

As described in more detail below, Process Analysis Module 62 is used to update the algorithm used by Demand-Based Module 60, based on feedback provided by Territory Users using Feedback Module 56. For example, the sample allocation calculated above may be multiplied by an “average allocation deviation ratio” calculated as:

Average Allocation Deviation Ratio=Trimmed Mean ((Rn)/n) where Rn=Sum of (Requested Allocation/Recommended Allocation) from previous allocations excluding the highest and lowest ratios.

n=Number of Ratios being taken into account.

Scripts-Based Module 64 creates allocation models based on scripts data for a specific prescriber (e.g., as stored in Scripts Database 28a5) that includes historical data regarding quantities of prescriptions written for each sample by the specific prescriber for a period of Z weeks. For example, an allocation model for a particular Territory T₁, for Medicine A for N weeks may be calculated as:

Sample Allocation=((N*(Y/Z)*F)−E

where:

| | |
|---|---|
| Y | Total samples of Medicine A received by the prescriber in Z weeks |
| | from all Territories |
| F | Quantity of Medicine A disbursed to the prescriber by Territory/X |
| X | Total scripts for Medicine A written by the prescriber in Z weeks |
| E | Estimated Inventory On Hand |

A User may specify a desired time period, desired samples and desired Territories, and Scripts-Based Module 64 may calculate quantities of the specified samples for the specified time period and the specified Territories based on scripts data, and may then create an allocation model for the specified time period and for the specified Territories.

Fixed Model 66 may allow a User to specify quantities of particular samples, for particular Territories, for particular time periods, and Fixed Model 66 may create an allocation model based on the specified information.

Referring again to FIG. 5, Display/Edit Module 54 enables a User to display, edit and/or approve an existing allocation model that is stored in Models Database 28a2, and then save the approved allocation model in Database 28a2. For example, as described above, Samples Operations 12 may use Display/Edit Module 54 to review, edit and/or approve data in an allocation model.

Feedback Module 56 enables a User to provide feedback regarding an allocation model. For example, as described above, Sales Team 18 may use Display/Edit Module 54 to view an allocation model, and then use Feedback Module 56 during an Allocation Window to provide feedback regarding the specified quantities.

In particular, using Feedback Module 56, a Territory User may agree that the specified quantities for each sample is correct, or may request more or less of one or more samples specified in the allocation model for their specific Territory. If the Territory User requests more or less of a sample, Feedback Module 56 may require that the User specify a reason for the modification (e.g., sufficient inventory on hand, updated usage rate data, and other similar reasons). In addition, Feedback Module 56 may calculate differences between original and revised quantities, and may provide this difference information to Analysis Module 62 (FIG. 4) to improve the process for calculating allocation models by Demand-Based Module 60.

Referring again to FIG. 3, Planning Module 58 allows Users to control how samples are made available to Sales Team 18. For example, Planning Module 58 may be used to create Allocation Schedules, described above. In addition, Planning Module 58 may be used to divide schedules into one or more sub-cycles during a particular time interval. Further, Planning Module 58 may be used to specify how allocations should be split up and made available for Ordering, and to partition the total allocations amongst multiple sub-cycles. For instance, if a 100 cases for a sample were allocated to a specific territory during a time interval having three sub-cycles, Planning Module 58 may be used to specify that 20% of the total allocation is available in the first sub-cycle, 30% in the second sub-cycle and the remaining in the last sub-cycle. As a result, only 20 (20% of 100 cases) cases of the product will be available for ordering during the first Order Window sub-cycle, 24 cases (30% of (100−20)) will be available for ordering in the second Order Window sub-cycle, and 56 cases (100−(20+24)) in the third Order Window sub-cycle.

Persons of ordinary skill in the art will understand that Allocation Module 30 may include more, fewer and/or different modules than the exemplary modules illustrated in FIG. 5.

### Order Module

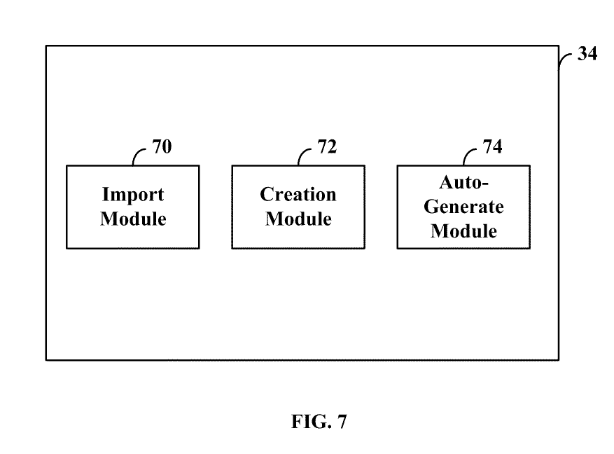

Referring again to FIG. 2, Order Module 34 enables Users to order samples based on the quantities specified in a reviewed allocation model stored in Transactions Database 28a7. Referring now to FIG. 7, an exemplary Order Module 34 is described. In particular, exemplary Order Module 34 includes Import Module 70, Creation Module 72 and Auto-Generate Module 74. In accordance with this invention, Orders may be imported, created, or automatically generated to the extent of quantities specified in a corresponding allocation model. For example, if an allocation model specifies that Territory 1 may order 100 units of Medicine A between January-April 2010, Territory 1 may create Orders for no more than 100 units of Medicine A during the specified time period. If Territory) wants to order additional units of Medicine A, the Samples Operation User has to reallocate additional units of Medicine A from the allocation belonging to Territory B. As Orders by a User are processed, Transaction Module 36 reduces the available allocation for the User by the amount ordered.

Referring again to FIG. 7, Import Module 70 enables a User to import an “externally-generated” Order. For example, a User may create an Order using a spreadsheet program that is separate from Samples Management System 10. Import Module 70 enables the User to import the externally generated Order into Samples Management System 10, and store the imported Order in Orders Database 28a6.

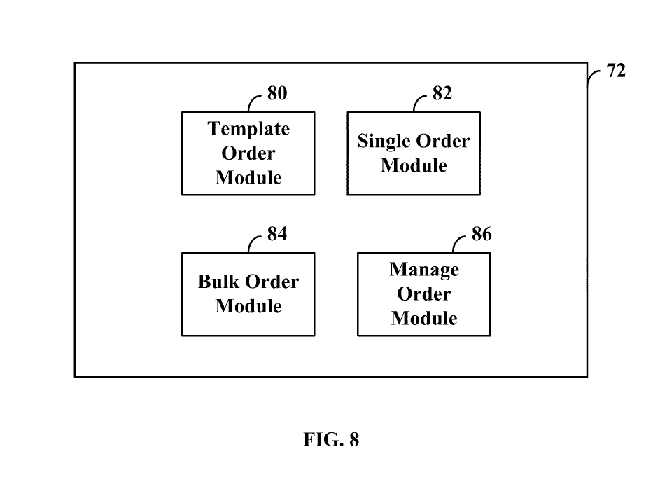

Creation Module 72 enables Users to create an Order within Samples Management System 10 and save the created Order to Orders Database 28a6. As illustrated in FIG. 8, exemplary Creation Module 72 may include Template Order Module 80, Single Order Module 82, Bulk Order Module 84 and Manage Order Module 86. Persons of ordinary skill in the art will understand that Creation Module 72 may include more, fewer or different modules than the ones illustrated in FIG. 8 for creating Orders.

Template Order Module 80 allows Users to create Orders based on pre-defined templates. For example, a system administrator may create a “New Hire Template” that includes predetermined quantities of one or more samples for a new sales representative. Persons of ordinary skill in the art will understand that other similar templates may be used.

Single Order Module 82 enables a User to create an Order for one or more samples for a single User. In accordance with an exemplary embodiment of this invention, a sales representative for a Territory will typically use Single Order Module 82 to create Orders for samples. In exemplary embodiments of this invention, Single Order Module 82 may enable a User to create a “Regular Order,” which is an Order that is created within a specified Order cycle for the User, and may also permit a User to create a “Supplementary Order,” which is an Order created outside the order cycle for the User.

Single Order Module 82 enables a User to select samples from a predetermined list of available samples that may be Ordered by the User, and also specify quantities of each selected sample. Additionally, Single Order Module 82 enables a User to create an Order based on a previous Order. Further, Single Order Module 82 enables a User to save a specified order as a Template for subsequent ordering use with Template Order Module 80. Moreover, Single Order Module 82 enables a User to order all remaining products and quantities available to the User as specified in the corresponding allocation model. Single Order Module 82 also enables a User to place an Order for immediate processing, or to schedule an Order for future processing.

Bulk Order Module 84 enables a User to create an Order for one or more samples for multiple Users. In accordance with an exemplary embodiment of this invention, a district manager or regional manager will typically use Bulk Order Module 84 to create Orders for samples for multiple Territories. As with Single Order Module 82, Bulk Order Module 84 may enable a User to select samples from a predetermined list of available samples that may be Ordered by the User, and also specify quantities of each selected sample. Additionally, Bulk Order Module 84 may enable a User to create an Order based on a previous Order. Further, Bulk Order Module 84 may enable a User to save a specified order as a Template for subsequent ordering use with Template Order Module 80. Moreover, Bulk Order Module 84 may enable a User to order all remaining products and quantities available to the User as specified in the corresponding allocation model. Bulk Order Module 84 also may enable a User to place an Order for immediate processing, or to schedule an Order for future processing.

Manage Order Module 86 enables a User to display details about Orders, such as tracking details, details about individual Orders, lists of multiple Orders, and other similar details. In some embodiments of this invention, Manage Order Module 86 may enable a User to cancel an Order, merge multiple Orders for a single User into a single Order, and split a single Order between multiple Warehouses 16 for fulfillment. Persons of ordinary skill in the art will understand that Manage Order Module 86 may enable Users to perform other display and Order management tasks.

Referring again to FIG. 7, Auto-Generate Module 74 enables a User to specify that the Samples Management System should automatically generate an order for the User when the inventory level for a specified sample (e.g., Medicine A) falls to a specified threshold (e.g., 4 weeks on hand). For example, Auto-Generate Module 74 may periodically monitor Inventory Database 28a3 to determine the inventory on hand for a Medicine A for Territory 1. If the inventory level of Medicine A for Territory 1 falls below the specified threshold, Auto-Generate Module 74 calculates the quantity of Medicine A that needs to be provided to Territory 1 for it to last X weeks, and generates an Order for the calculated quantity. Auto-Generate Module 74 also may determine the quantities of other samples products that are in the possession of Territory 1, but above the specified threshold, that needs to be provided to Territory 1 for it to last X weeks, and may generate Order for those other samples products.

### Transaction Module

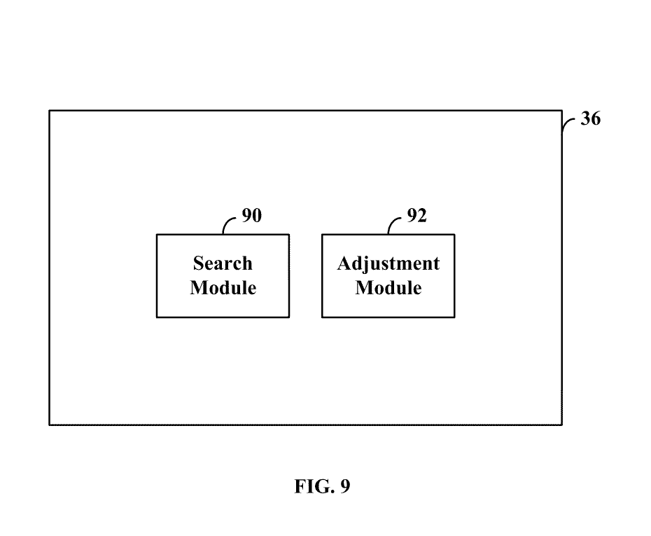

Referring again to FIG. 2, Transaction Module 36 enables Users to view and manage transaction records in samples management system 10 for reconciliation purposes. Referring now to FIG. 9, an exemplary Transaction Module 36 is described. In particular, exemplary Transaction Module 36 includes Search Module 90 and Adjustment Module 92. In accordance with this invention, a “Transaction Record” is a record of an activity that occurs in samples management system 10, each of which is stored in Transactions Database 28a7. Exemplary Transaction Records may include disbursements (e.g., deliveries of samples to Prescribers 20), shipments (e.g., shipments of samples from Warehouse 16 to Territories), inventory records, returns (e.g., expiring/damaged samples that must be returned), theft/loss, transfer-in and transfer-out records (e.g., transfers between Territories), adjustment records, and other similar records.

Search Module 90 enables Users to search for Transaction Records within samples management system 10. Exemplary search criteria may include transaction number, Territory, Region, District, Sales Force, date range, transaction type, transaction status, product name and/or lot number, employee name, and/or other similar search criteria.

Adjustment Module 94 enables Users to edit/revise Transaction Records stored in Transactions Database 28a7. In an exemplary embodiment of this invention, a result of adjusting a Transaction Record is an “Adjustment Record” that overrides (but does not replace) the content of the original Transaction Record that was adjusted. The original Transaction Record may be retained for informational use. In accordance with exemplary embodiments of this invention, Adjustment Module 94 enables Users to adjust Transaction Records individually, or as a group. Also in accordance with exemplary embodiments of this invention, Adjustment Module 94 may provide approval mechanisms whereby Adjustment Records may require approval (e.g., by a User's supervisor, etc.) before the Adjustment Record is completed.

### Reconciliation Module

Sales representatives typically are required to report inventory during applicable Inventory Submission Intervals. Referring again to FIG. 2, Reconciliation Module 38 enables Users to reconcile a sales representative's reported inventory with the electronic activity that was tracked for the sales associate for a given time period (e.g., from the prior Inventory Submission Interval to the current Inventory Submission Interval).

Reconciliation Module 38 automatically initiates a reconciliation process when a reconcilable inventory transaction is imported into the system. The reconciliation process generates reconciliation information from the last inventory received for the field user to the most current inventory that was imported for that field user. Additionally, reconciliation module also allows users to generate ad-hoc reconciliation reports. The reconciliation information is acted upon by Samples Operations 12 Users who identify reconciliations that are above the acceptable variance thresholds and investigate the reasons behind the out of bound conditions.

In an exemplary embodiment of this invention, Reconciliation Module 38 performs the inventory reconciliation based on: (1) the beginning inventory for the period; (2) the ending inventory for the period; (3) the number of samples received by the sales representative as shipments during the interval between the beginning inventory and the ending inventory (the “Inventory Interval”); (4) the number of samples disbursed by the sales representative to Prescribers 20 during the Inventory Interval; (5) the number of samples received as transfer-in from another sales representative during the Inventory Interval; (6) the number of samples given out as transfer-out to another sales representative during the Inventory Interval; (7) the number of samples returned for destruction during the Inventory Interval; (8) the number of samples reported as lost or stolen during the Inventory Interval; and (9) adjustments to account for extraneous errors.

In exemplary embodiments of this invention, Reconciliation Module 38 provides lists of reconciled and un-reconciled inventories. Access to the lists may be limited based on User status. For instance, a district manager may only view reconciliation information for sales representatives in their district and themselves. A particular sales representative would only be able to view their own reconciliation information.

In exemplary embodiments of this invention, Reconciliation Module 38 retains a history of previous reconciliations conducted for each sales representative. When a User selects a specific inventory reconciliation, Reconciliation Module 38 may provide a real time data grid that displays the product count summaries for each transaction type and each product for that particular sales representative. Each summary count field is “hot,” and when the User clicks on it the underlying transactional information that was used for the summary count is displayed. The user can then make any transaction adjustments, and the adjusted quantities are reflected in the reconciliation totals by refreshing the reconciliation totals. In accordance with exemplary embodiments of this invention, a reconciliation can be considered UnReconciled, Reconciled, Reconciled—Forced. When an inventory period is tagged as Reconciled it indicates that the reconciliation for that inventory time period can be considered closed and no further reconciliation activity is required.

### Reports Module

Referring again to FIG. 2, Reports Module 40 enables Users to create and view reports. In exemplary embodiments of this invention, Users may create and view predefined reports, and also may use a reports builder tool to create and view custom reports using data available in Database 28. In addition, Reports Module 40 enables Users to configure the method of transmission of their reports and the frequency with which reports are generated. Additionally, Users may save generated reports, and may specify the output file format for saved reports. Further, Reports Module 40 may retain a database of historical reports generated by Reports Module 40.

### Analytics Module

Referring again to FIG. 2, Analytics Module 42 provides interactive tools that enable Users to analyze data in Database 28. For example, Analytics Module 42 may be used to analyze and display data regarding allocation models, Orders, Transaction Records, Inventory Reconciliations and operational work data that is captured within samples management system 10. For example, Analytics Module 42 enables Users to create, manipulate and display analytical charts regarding shipments, disbursements, returns, transfers, theft/loss, inventory, estimated inventory on hand, allocations, and other similar data.

### Dashboard Module

Referring again to FIG. 2, Dashboard Module 44 provides a “Performance Dashboard” that provide a summary view of various metrics associated with samples management system 10. In an exemplary embodiment in accordance with this invention, Dashboard Module 44 provides a combination of User-selectable Alerts, Metrics and Charts to provide a snapshot summary of the User's current operational, tactical and strategic business process goals.

For example, performance charts may be grouped by underlying Samples Management Subprocesses Reconciliation Management, Allocation Management, Order & Shipment Management, Returns Management, Theft/Loss Management, and Inventory On-Hand Management. Persons of ordinary skill in the art will understand that more, fewer or different performance charts may be used.

In exemplary embodiments of this invention, User may select multiple metrics and charts for each sub-process, and may drill down from the highest level summary data to the lowest level summary data.

### Alerts Module

Referring again to FIG. 2, Alerts Module 46 provides alerts and notifications (collectively referred to as “Alerts”) to inform Users about particular events that require attention. Alerts Module 46 may send Alerts to individual Users, or to groups of multiple Users. Alerts Module 46 enables Users to manage and save Alerts. Alerts Module 46 may include predetermined Alerts, and also may enable Users to create custom alerts.

In accordance with this invention, Alerts may be transactional and process related. Alerts Module 46 generates transactional alerts based on a specific transaction. For example, an Alert may be generated if a particular shipment was not acknowledged for a predetermined time period. In contrast, Alerts Module 46 generates process-based alerts when a certain process metric exceeds acceptable Upper or Lower thresholds. Table 2 provides examples of various transactional and process alerts that may be generated by Alerts Module 44 for various alert types.

TABLE 2
Exemplary Transactional and Process Alerts

| Alert Type | Transactional Alert | Process Alert |
|---|---|---|
| Shipment | Shipment has not been | Percentage increase in |
| | acknowledged as received for | unacknowledged shipments for |
| | more than a predetermined | more than X days exceeds a |
| | number of days. | predetermined % value. |
| Transfer | Transfer to a Non-Active | Products transferred in current |
| | Employee | month greater than previous |
| | | month by X %. |
| Return | Return not acknowledged by | Products returned in current |
| | Destruction Facility in over X | month greater than previous |
| | days. | month by X %. |
| Disbursement | Disbursement made on a non- | Percent increase or decrease in |
| | business day. | disbursed quantity for a product |
| | | month over month. |
| Theft-Loss | Theft/Loss transaction received | Multiple theft/loss reported in last |
| | after closed inventory | X months. |
| | reconciliation period. | |
| Standalone- | Stand-alone adjustment received | Total number of stand-alone |
| Adjustment | after closed inventory | adjustments for a particular region |
| | reconciliation period. | higher than organization average |
| | | by X %. |
| Inventory | Inventory has duplicate | Total inventory on hand exceeds |
| | product/lot lines. | limit of X weeks on hand. |
| Adjustment | Adjustment made to a transaction | Number of adjustments made to |
| | in a closed inventory period. | the same transaction exceeds X |
| | | times. |
| Reconciliation | Inventory submitted is dated prior | Percentage of inventories above |
| | to the latest reconciled period. | threshold variance exceeds X %. |

In accordance with exemplary embodiments of this invention, Users may click on alerts to display the underlying details within the alert. In addition, in the case of transactional alerts, users may also edit the transaction that generated the alert to address the alert condition.

### System Implementation

Systems and methods in accordance with this invention may be implemented in software (e.g., firmware), hardware, or a combination of software and hardware. In exemplary embodiments, systems and methods in accordance with this invention may be implemented as a computer-implemented method, system, and computer program product. In particular, this invention may be implemented within a network environment (e.g., the Internet, a wide area network (“WAN”), a local area network (“LAN”), a virtual private network (“VPN”), etc.), or on a stand-alone computer system. In the case of the former, communication throughout the network can occur via any combination of various types of communications links. For example, the communication links may comprise addressable connections that may utilize any combination of wired and/or wireless transmission methods. Where communications occur via the Internet, connectivity could be provided by conventional TCP/IP sockets-based protocol, and an Internet service provider could be used to establish connectivity to the Internet.

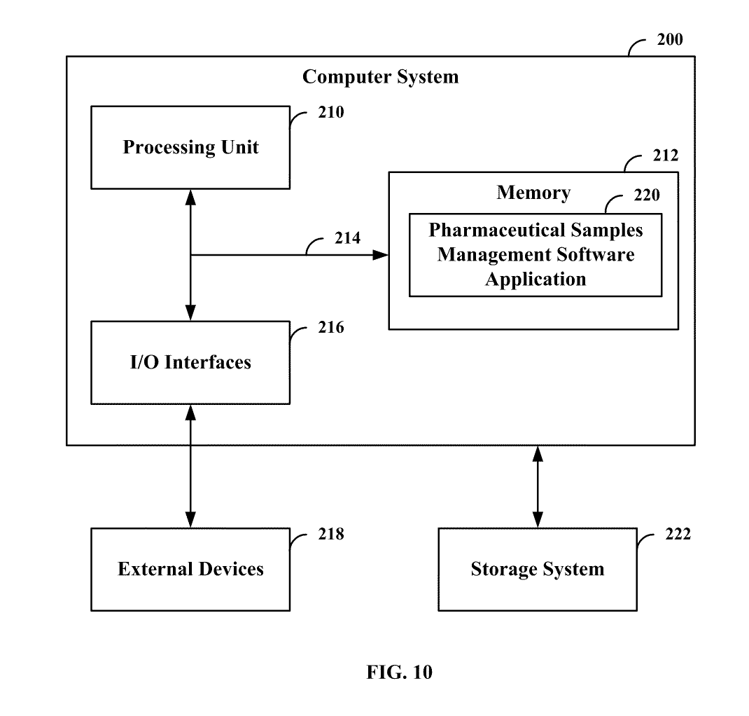

For example, as shown in FIG. 10, the present invention may be implemented on a computer system, such as computer system 200 that includes a processing unit 210, a memory 212, a bus 214, input/output (“I/O”) interfaces 216 and external devices 218. Processing unit 210 may be a computer or processing unit of any type that is capable of performing the functions described herein. Memory 212 is capable of storing a set of machine readable instructions (i.e., computer software) executable by processing unit 210 to perform the desired functions. Memory 212 is any type of computer-readable media or device for storing information in a digital format on a permanent or temporary basis, such as, e.g., a magnetic media, optical media, flash memory, random access memory, or other similar memory.

In particular, memory 212 includes a pharmaceutical samples management software application 220, which is a software program that contains computer-executable program code. Software application 220 contains an ordered listing of computer-executable instructions for implementing functions of the present invention. Alternatively, pharmaceutical samples management software application 220 may be stored on storage system 222. Processing unit 210 executes the pharmaceutical samples management software application 220. While executing computer program code 220, processing unit 210 can read and/or write data to/from memory 212, storage system 222 and/or I/O interfaces 216. Bus 214 provides a communication link between each of the components in computer system 200. External devices 218 can comprise any devices (e.g., keyboard, pointing device, display, etc.) that enable a user to interact with computer system 200 and/or any devices (e.g., network card, modem, etc.) that enable computer system 200 to communicate with one or more other computing devices.

Computer system 200 may include two or more computing devices (e.g., a server cluster) that communicate over a network to perform the various process steps of the invention. Embodiments of computer system 200 can comprise any specific purpose computing article of manufacture comprising hardware and/or computer program code for performing specific functions, any computing article of manufacture that comprises a combination of specific purpose and general purpose hardware and/or software, or the like. In each case, the program code and hardware can be created using standard programming and engineering techniques, respectively.

Moreover, processing unit 210 can comprise a single processing unit, or can be distributed across one or more processing units in one or more locations, e.g., on a client and server. Similarly, memory 212 and/or storage system 222 can comprise any combination of various types of data storage and/or transmission media that reside at one or more physical locations. Further, I/O interfaces 216 can comprise any system for exchanging information with one or more external devices 218. In addition, one or more additional components (e.g., system software, math co-processing unit, etc.) not shown in FIG. 8 can be included in computer system 200.

Storage system 222 may include one or more storage devices, such as a magnetic disk drive or an optical disk drive. Alternatively, storage system 222 may include data distributed across, for example, a LAN, WAN or a storage area network (“SAN”) (not shown). Although not shown in FIG. 10, additional components, such as cache memory, communication systems, system software, etc., may be incorporated into computer system 200.

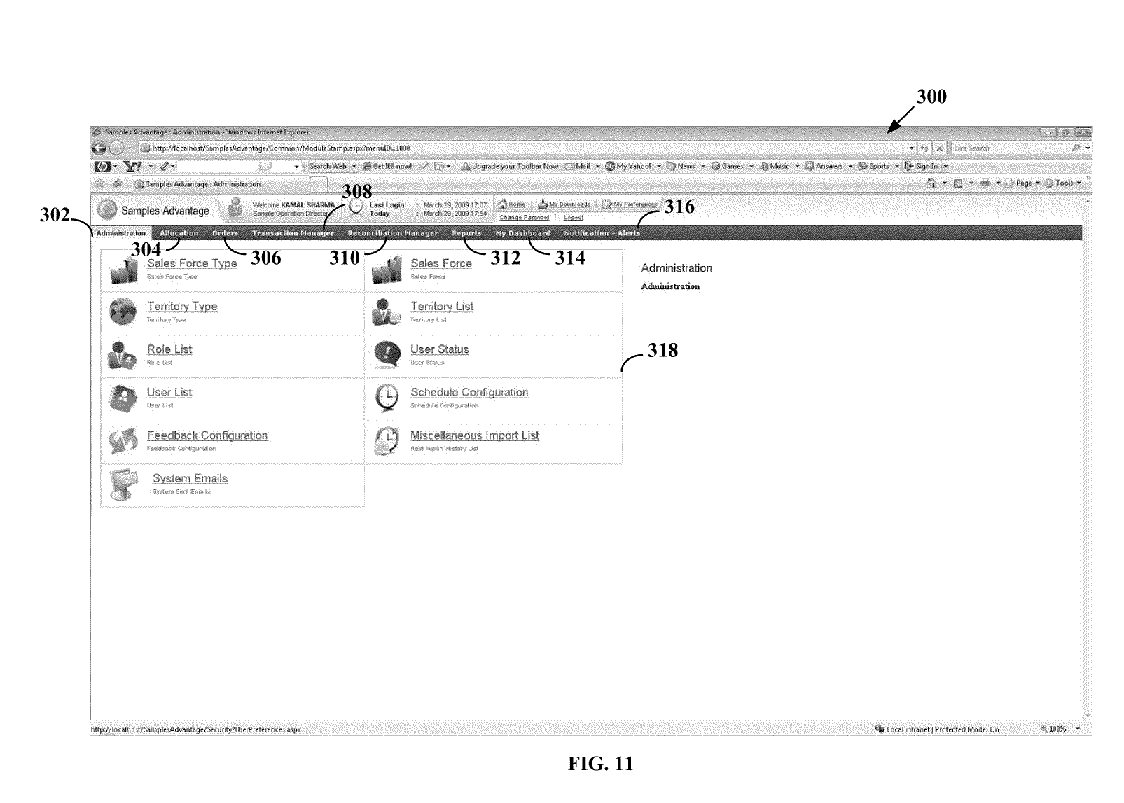

As mentioned above, samples management system 10 includes a user interface that enables Users to interact with Modules 30-46 to design, implement and monitor samples programs. An exemplary user interface which allows Users to design, implement and monitor samples programs is described below with reference to FIGS. 11-18.

Referring now to FIG. 11, an exemplary user interface 300 is shown. User interface 300 includes User-selectable tabs for selecting and switching between displays related to the following functions: Administration 302, Allocation 304, Orders 306, Transaction Manager 308, Reconciliation Manger 310, Reports 312, Dashboard 314 and Notification-Alerts 316. Persons of ordinary skill in the art will understand that users interfaces in accordance with this invention may include more, fewer or different functional tabs than the ones depicted in FIG. 11.

As shown in FIG. 11, administration tab 302 includes a User-selectable work area 318 for use by systems administrators for selecting and controlling various system functions implemented by Administration Module 28, described above. For example, system administrators may specify sales forces and sales force types, specify territories and territory types, generate User lists, define User roles, and set User status, configure schedules and feedback, and set various other system configuration settings. Persons of ordinary skill in the art will understand that administration tabs in accordance with this invention may include more, fewer or different administrator settings than the ones depicted in FIG. 11.

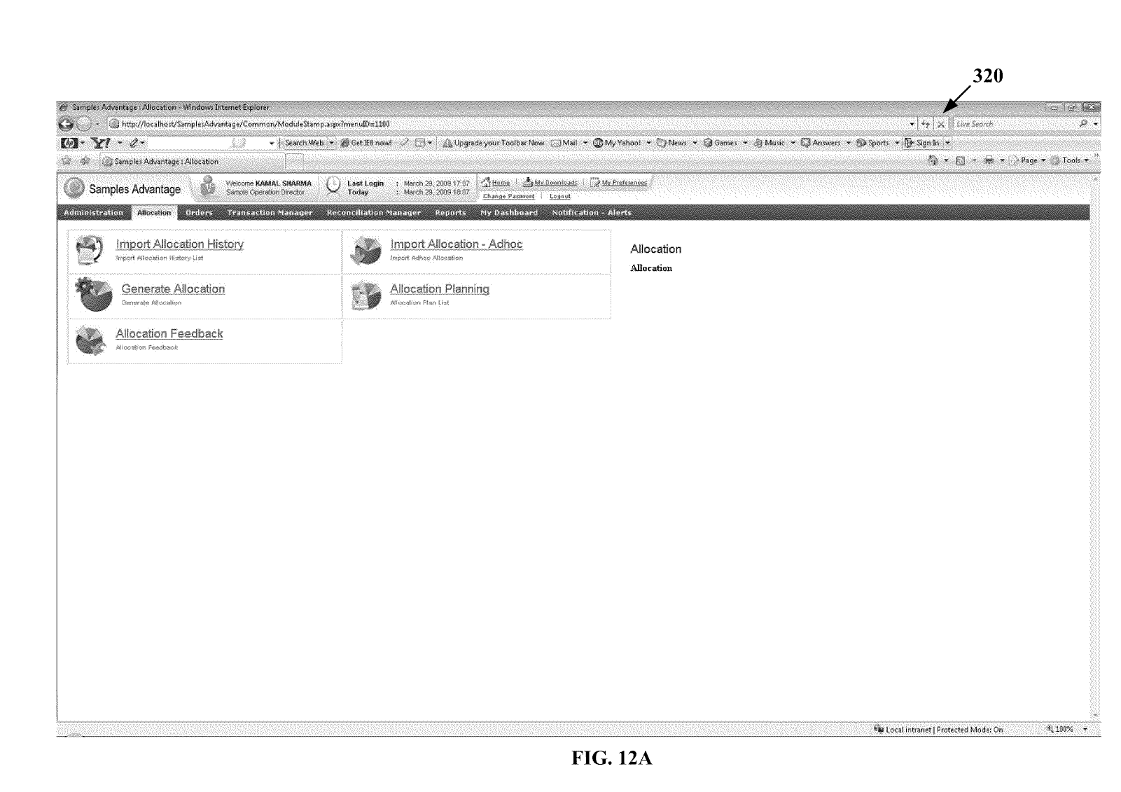

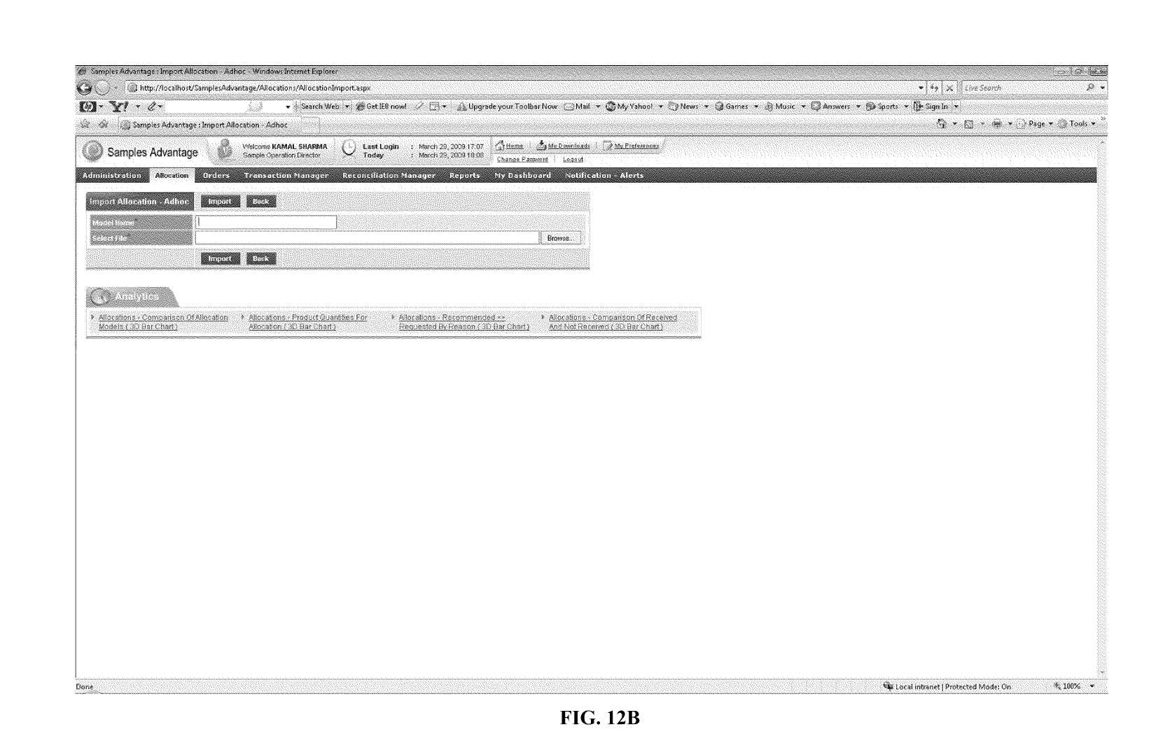

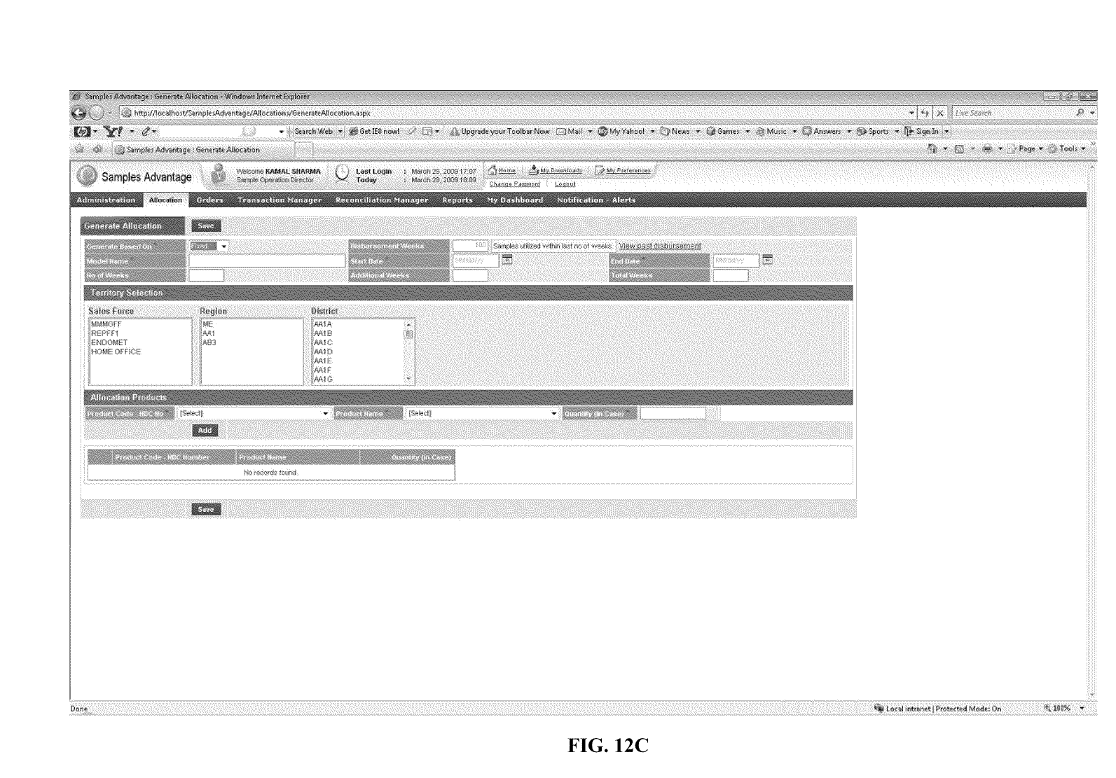

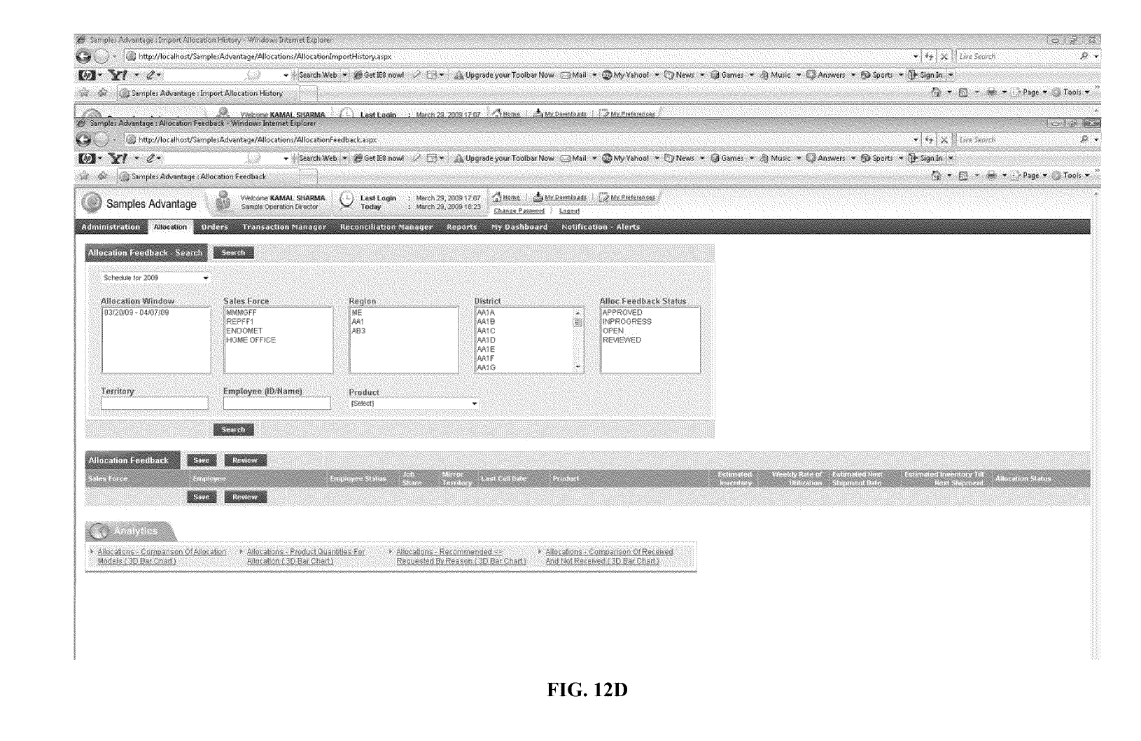

Referring now to FIGS. 12A-D, an exemplary Allocation user interface 320 is described. In particular, Allocation user interface 320 enables Users to interact with Allocation Module 30 to create allocation models, and to display, provide feedback, edit and approve quantities of samples specified in allocation models. As shown in FIGS. 12A-12D, various User-selectable fields enable Users to import allocations, generate allocations, display lists of imported allocations, provide allocation feedback, and display information about allocation planning Persons of ordinary skill in the art will understand that Allocation user interfaces in accordance with this invention may include more, fewer or different settings than the ones depicted in FIGS. 12A-12D.

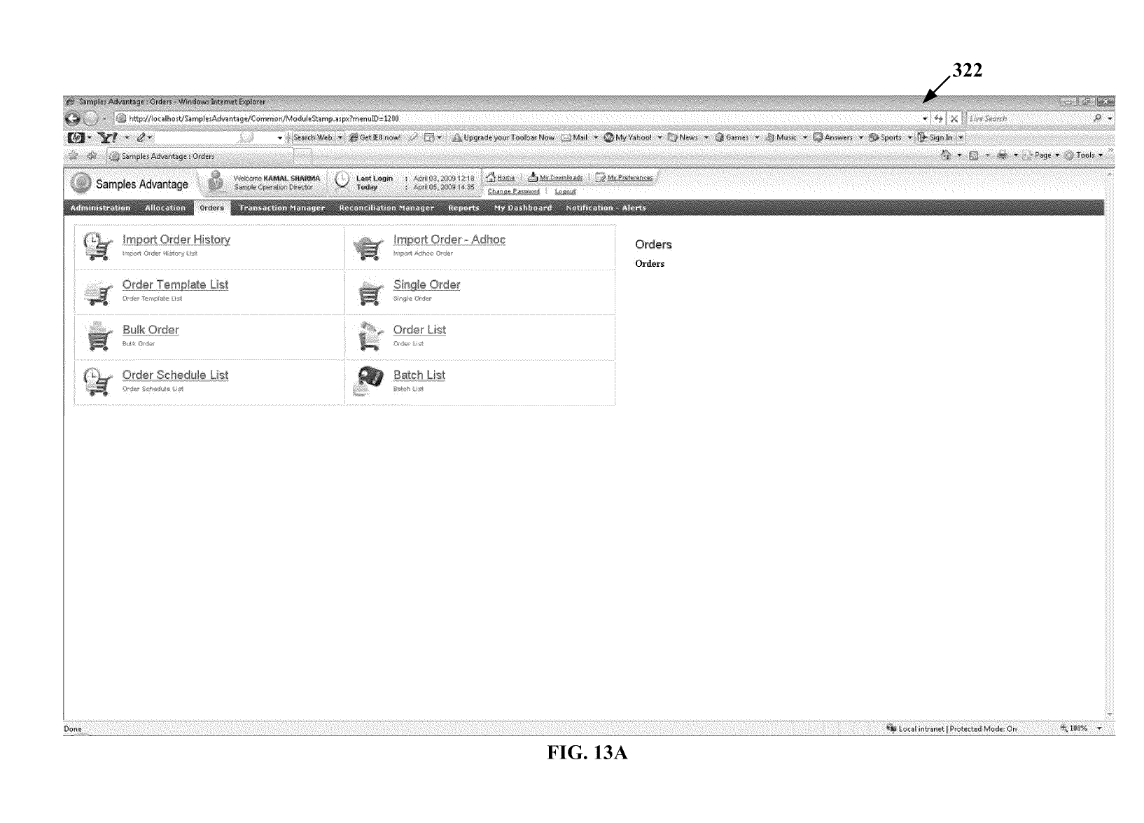

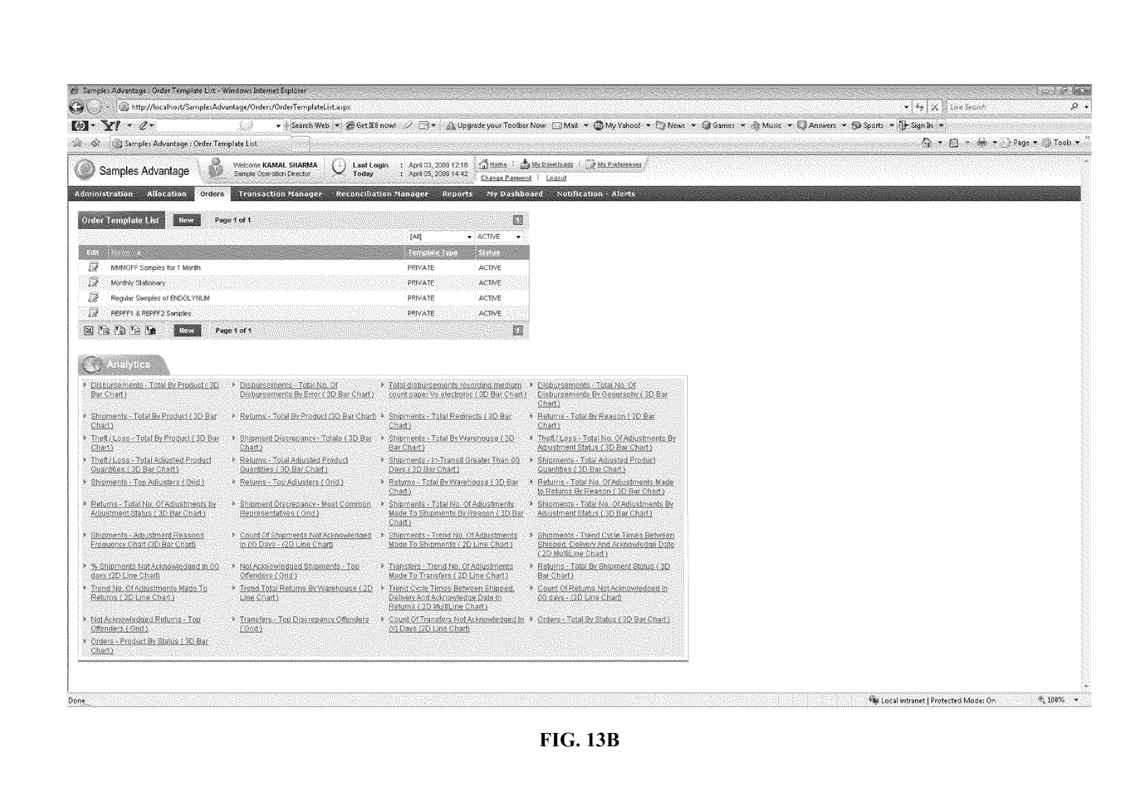

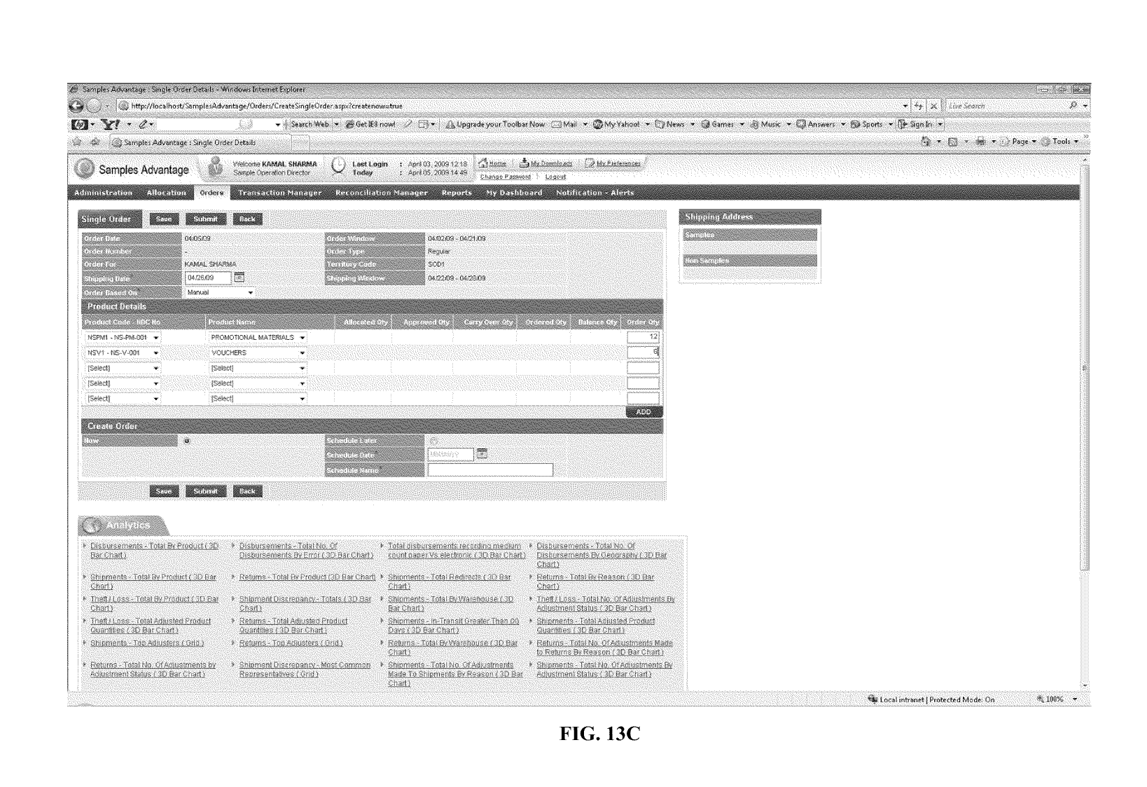

Referring now to FIG. 13A-13C, an exemplary Orders user interface 322 is described. In particular, Orders user interface 322 enables Users to interact with Order Module 34 to order samples based on the quantities specified in a reviewed allocation model stored in Transactions Database 28a7. As shown in FIGS. 13A-13C, various User-selectable fields enable Users to import orders, generate orders (single and bulk), display lists of imported orders, and display information about order schedules. Persons of ordinary skill in the art will understand that Orders user interfaces in accordance with this invention may include more, fewer or different settings than the ones depicted in FIGS. 13A-13C.

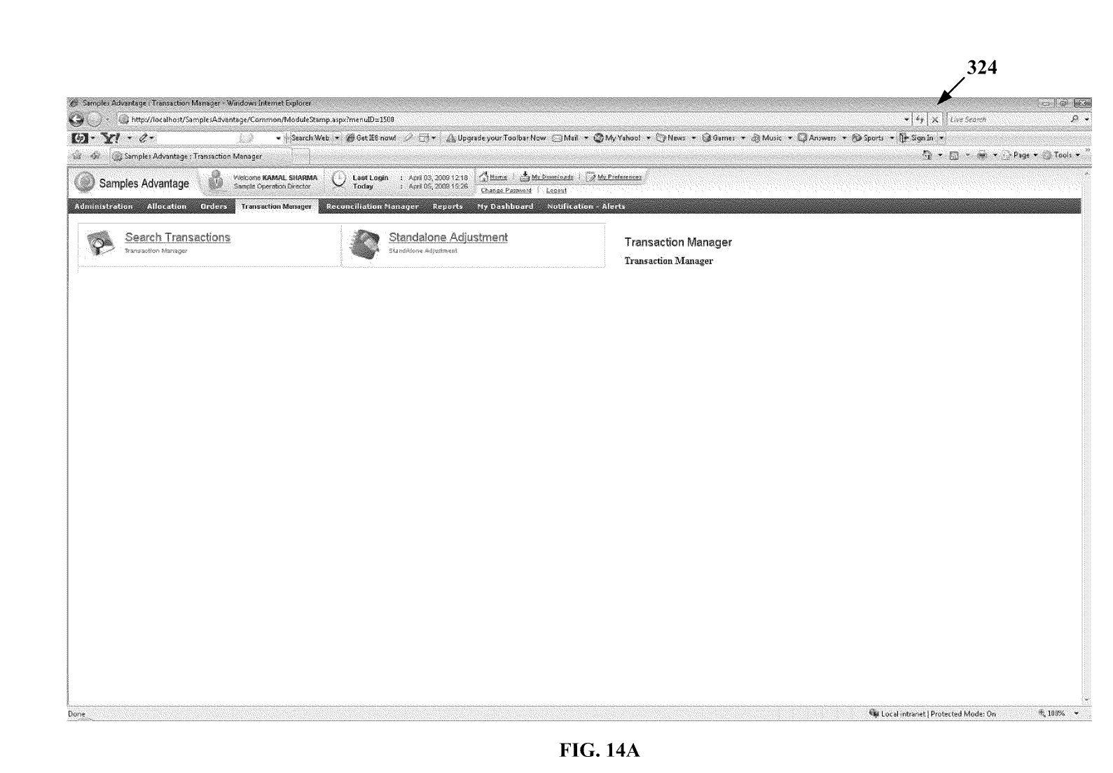

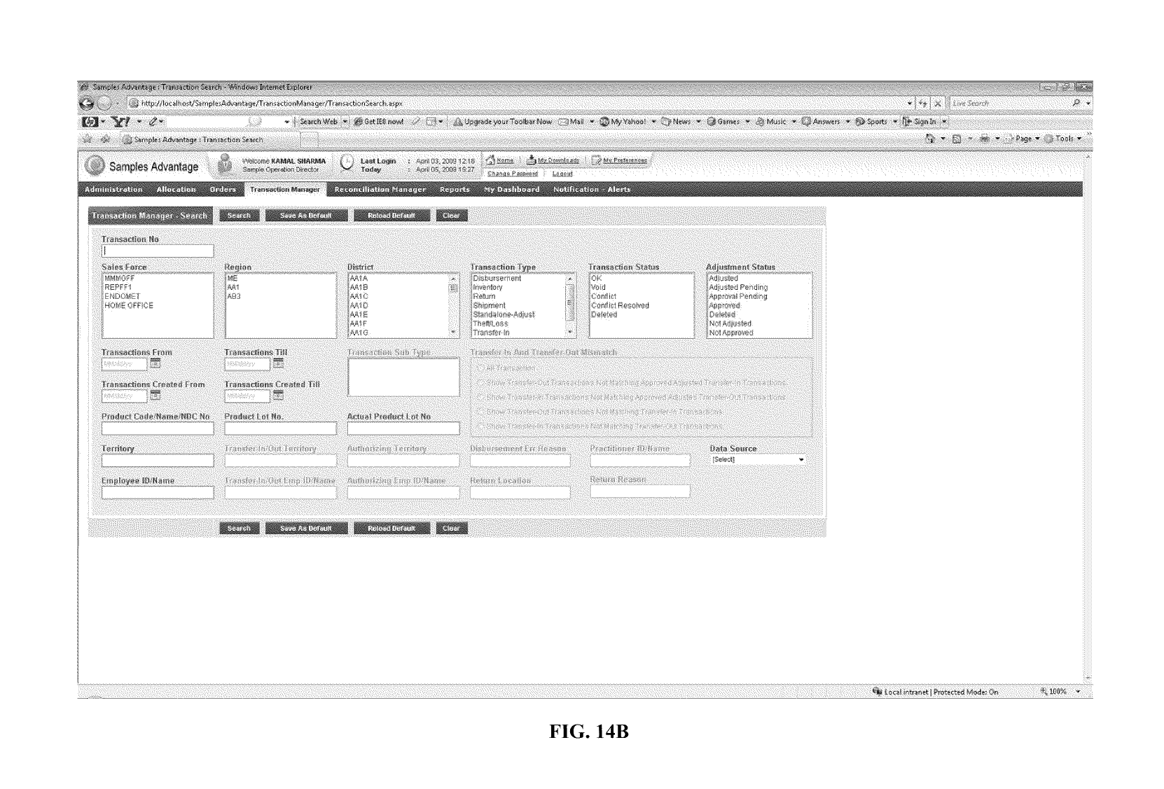

Referring now to FIG. 14A-14B, an exemplary Transactions Manager user interface 324 is described. In particular, Transactions Manager user interface 324 enables Users to interact with Transaction Module 36 to view and manage transaction records in samples management system 10 for reconciliation purposes. As shown in FIGS. 14A-14B, various User-selectable fields enable Users to search for Transaction Records within samples management system 10, and to edit/revise Transaction Records stored in Transactions Database 28a7. Persons of ordinary skill in the art will understand that Transactions Manager user interfaces in accordance with this invention may include more, fewer or different settings than the ones depicted in FIGS. 14A-14B.

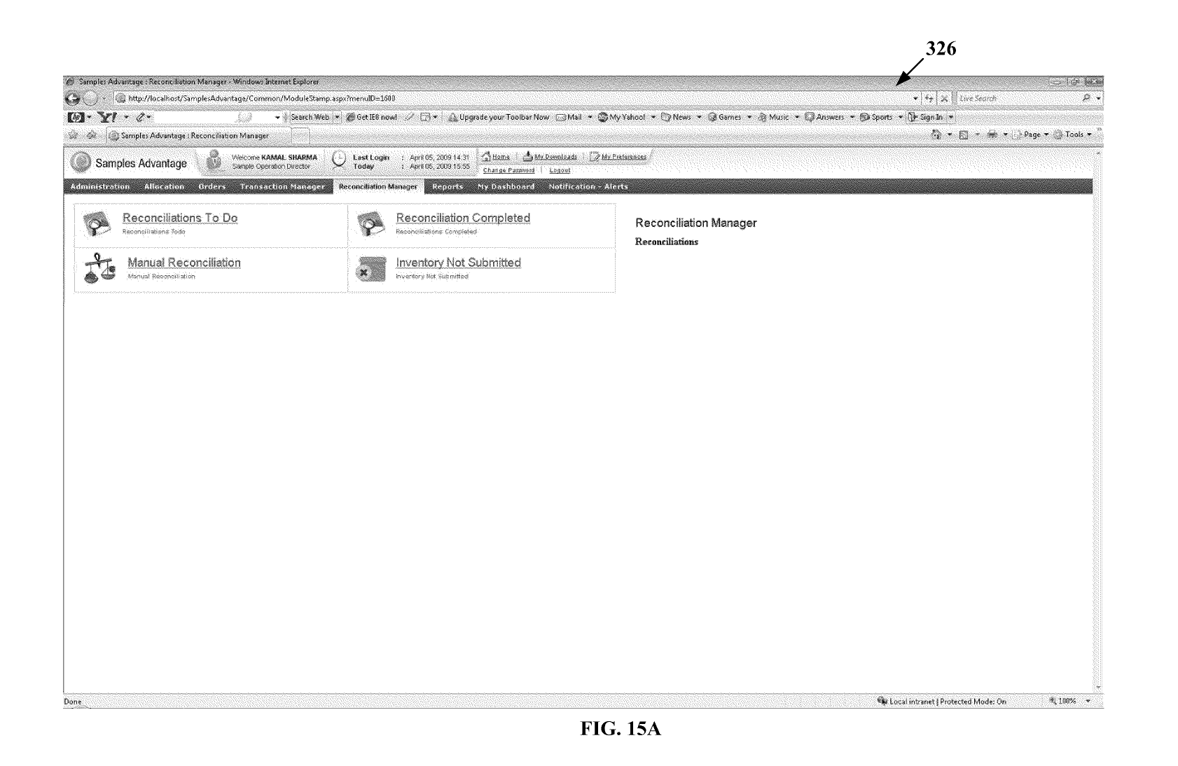

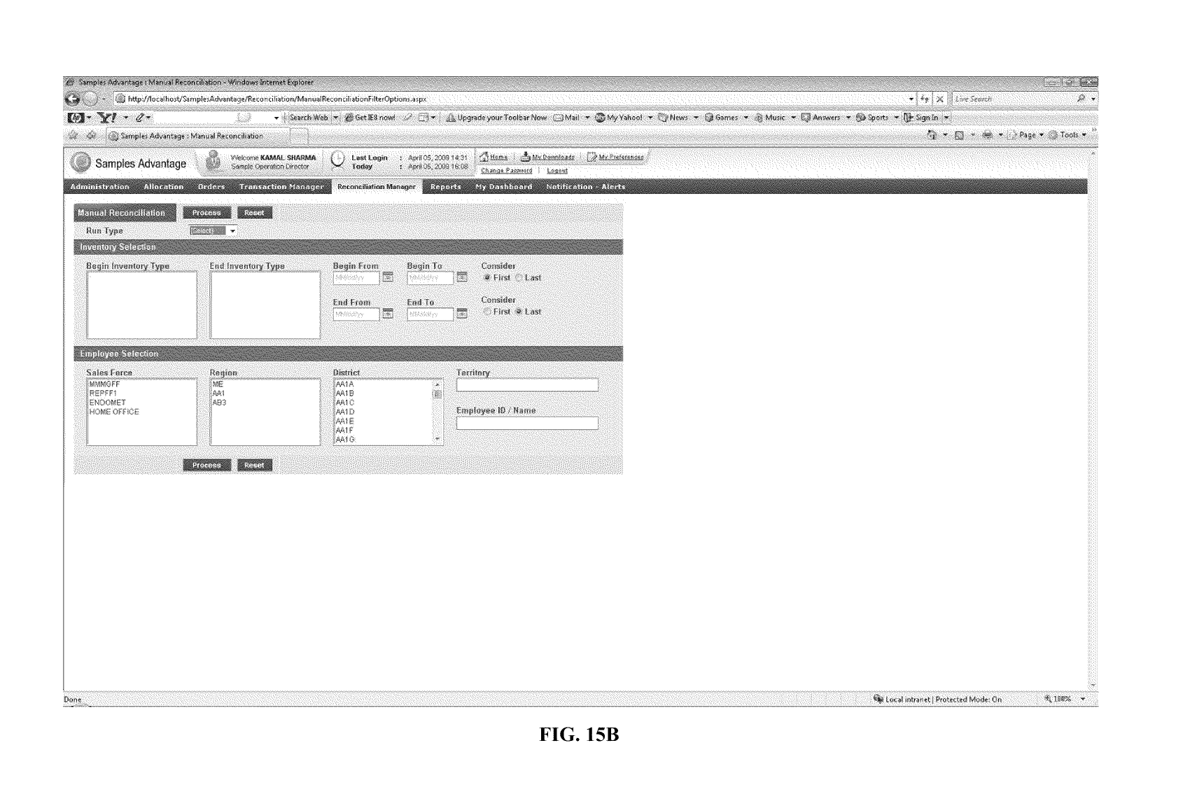

Referring now to FIG. 15A-15B, an exemplary Reconciliation Manager user interface 326 is described. In particular, Reconciliation Manager user interface 326 enables Users to interact with Reconciliation Module 38 to reconcile a sales representative's reported inventory with the electronic activity that was tracked for the sales associate for a given time period. As shown in FIGS. 15A-15B, various User-selectable fields enable Users to display reconciliation to-do lists, display lists of completed reconciliations, perform manual reconciliations, and display lists of inventory not submitted. Persons of ordinary skill in the art will understand that Reconciliation Manager user interfaces in accordance with this invention may include more, fewer or different settings than the ones depicted in FIGS. 15A-15B.

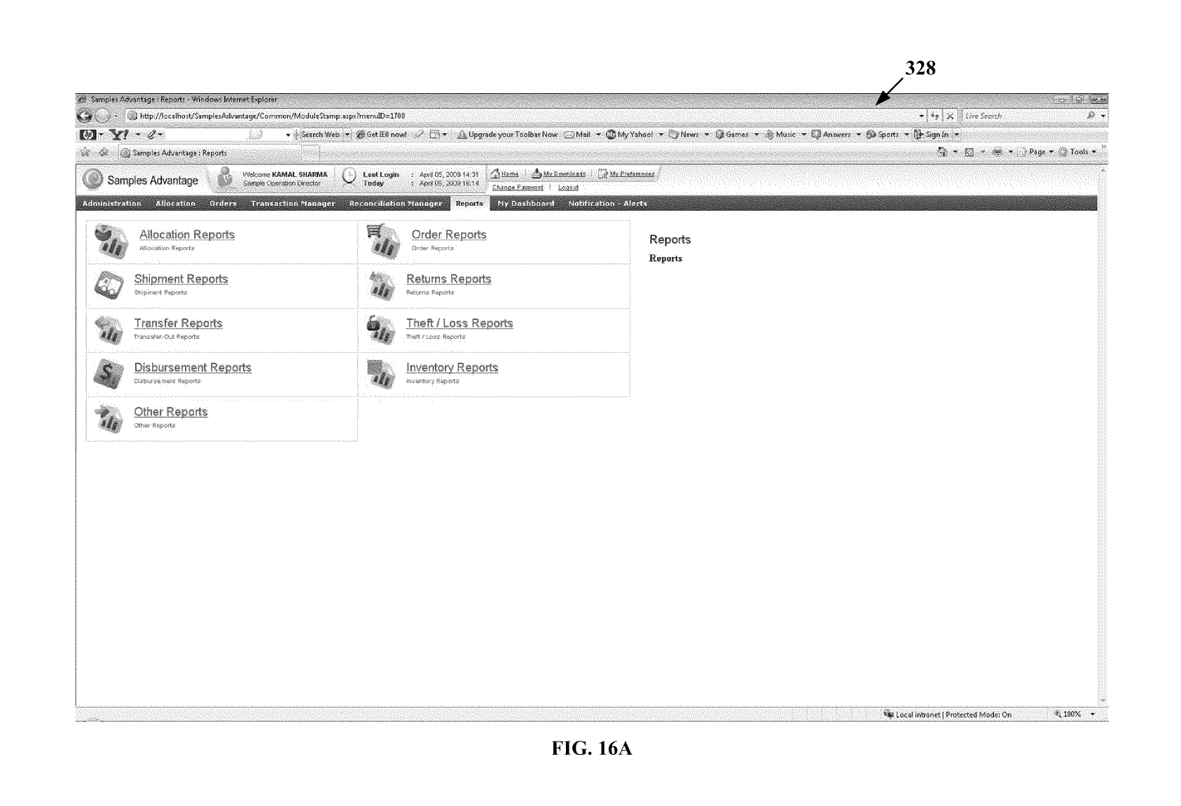

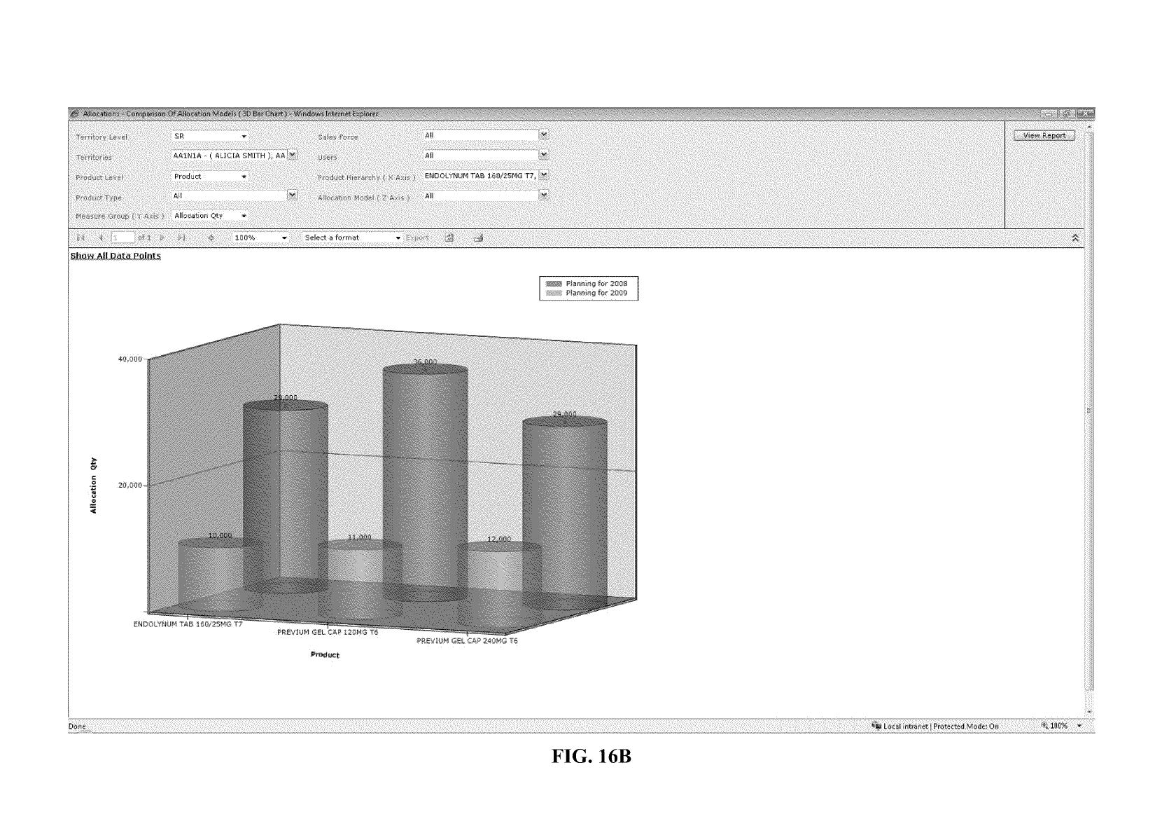

Referring now to FIG. 16A-16B, an exemplary Reports user interface 328 is described. In particular, Reports user interface 328 enables Users to interact with Reports Module 40 to create and view reports. As shown in FIGS. 16A-16B, various User-selectable fields enable Users to generate reports regarding allocations, orders, shipments, returns, transfers, theft/loss, disbursements, inventories, and other similar. Persons of ordinary skill in the art will understand that Reports user interfaces in accordance with this invention may include more, fewer or different settings than the ones depicted in FIGS. 16A-16B.

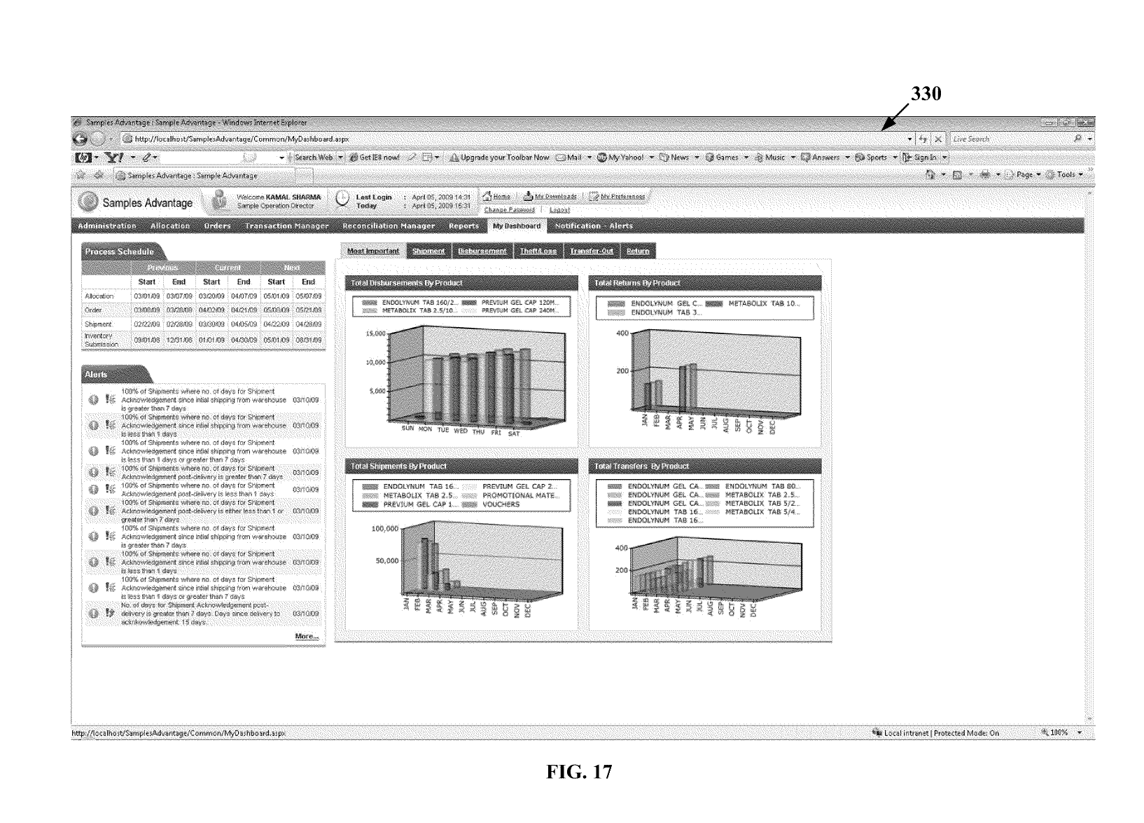

Referring now to FIG. 17, an exemplary My Dashboard user interface 330 is described. In particular, My Dashboard user interface 330 enables Users to view a summary view of various metrics associated with samples management system 10, as created by Dashboard Module 44. As shown in FIG. 17, My Dashboard user interface 330 provides a combination of User-selectable Alerts, Metrics and Charts to provide a snapshot summary of the User's current operational, tactical and strategic business process goals. Persons of ordinary skill in the art will understand that My Dashboard user interfaces in accordance with this invention may include more, fewer or different settings than the ones depicted in FIG. 17.

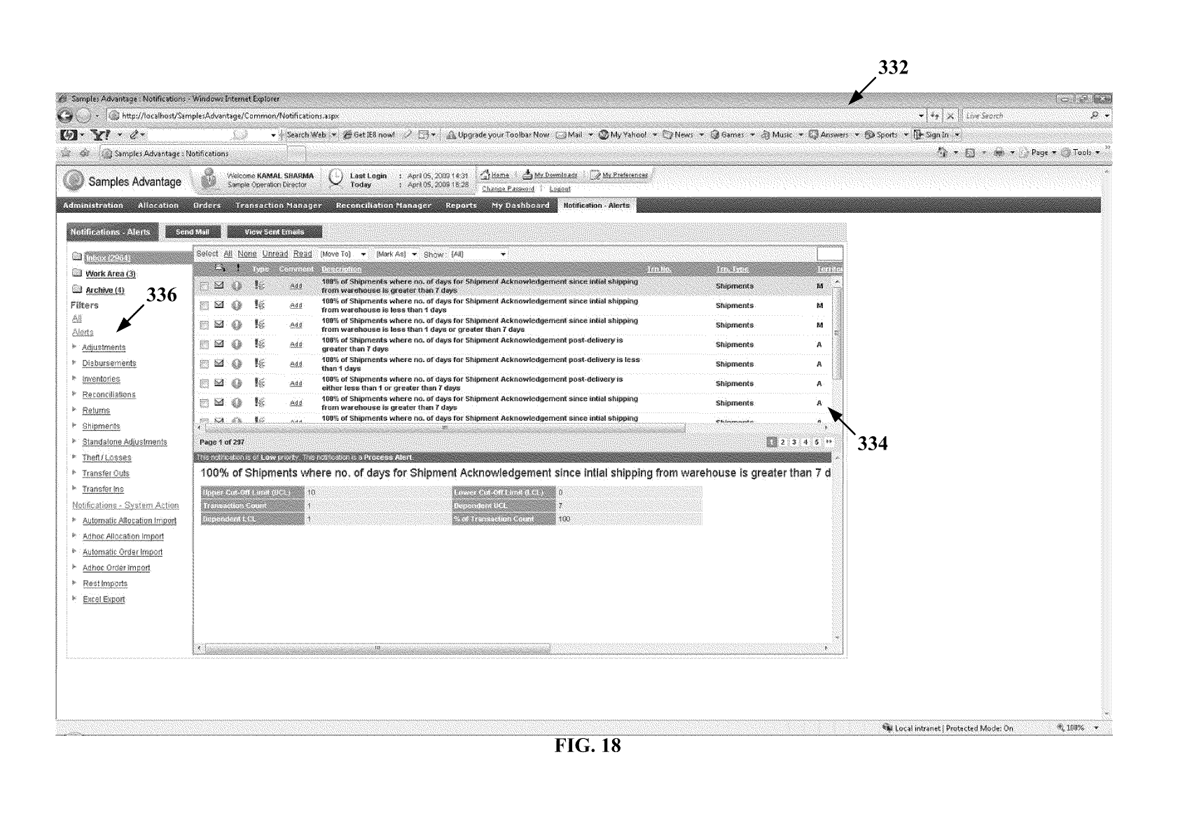

Referring now to FIG. 18, an exemplary Notification-Alerts user interface 332 is described. In particular, Notification-Alerts user interface 332 enables Users to view Alerts provided by Alerts Module 46. As shown in FIG. 18, Notification-Alerts user interface 332 includes a window 334 for displaying alerts and notifications, and also may include one or more filters 336 for selectively displaying some, all, or specific types of alerts and notifications. Persons of ordinary skill in the art will understand that Notification-Alerts user interfaces in accordance with this invention may include more, fewer or different settings than the ones depicted in FIG. 18.

The foregoing merely illustrates the principles of this invention, and various modifications can be made by persons of ordinary skill in the art without departing from the scope and spirit of this invention.

1. A system for pharmaceutical samples management, the system comprising:
an allocation module adapted to allow a user to create allocation models for pharmaceutical samples, wherein an allocation model specifies quantities of pharmaceutical samples that are allocated amongst a sales team for specified time periods;
an order module adapted to allow the user to order pharmaceutical samples based on an allocation model;
a transaction module adapted to allow the user to monitor a transaction in the system;
a reconciliation module adapted to allow the user to perform samples inventory reconciliation;
a reports module adapted to allow the user to obtain reports regarding the system;
an analytics module adapted to allow the user to obtain analytical information regarding the system;
a dashboard module adapted to allow the user to display data regarding the system; and
an alerts module adapted to provide alert information regarding the system.

2. The system of claim 1, further comprising a database that includes:
an operational database for storing data for transaction processing data;
a data warehouse database for storing data extracted from the operational database in a form suitable for use by the reports module and the analytics module;
a system control database for storing system configuration elements, such as database connectivity, system folder structures and files names, web server configuration details; and
an import/export database for storing data imported from and/or exported to external sources and the samples management system.

3. The system of claim 1, further comprising an administration module adapted to allow the user to specify allocation schedules, order schedules, shipping schedules and inventory schedules.

4. The system of claim 1, wherein the allocation module is further adapted to allow the user to display, provide feedback, edit and approve quantities of samples specified in allocation models.

5. The system of claim 1, wherein the allocation module further comprises:
an import module adapted to allow the user to import externally-generated allocation models;
a creation module that includes:
a demand-based module adapted to create allocation models based on historical samples data for each sales team;
a scripts-based module adapted to create allocation models based on scripts data for a specific prescriber; and
a fixed module adapted to create an allocation model based on a specified quantity of particular samples, for particular sale teams, for particular time periods;
a display/edit module adapted to allow the user to display, edit and/or approve an existing allocation model;
a feedback module adapted to allow the user to provide feedback regarding an allocation model; and
a planning module adapted to allow the user to control how samples are made available to a sales team.

6. The system of claim 1, wherein the order module further comprises:
an import module adapted to allow the user to import an externally-generated order;
a creation module that includes:
a template order module adapted to allow the user to create orders based on pre-defined templates;
a single order module adapted to allow the user to create an order for one or more samples for a single user;
a bulk order module adapted to allow the user to create an order for one or more samples for multiple users;
a manage order module adapted to allow the user to display details about orders, such as tracking details, details about individual orders, lists of multiple orders; and
an auto-generate module adapted to allow the user to specify that the system should automatically generate an order for the user when an inventory level for a specified sample falls to a specified threshold.

7. The system of claim 1, wherein the transaction module further comprises:
a search module adapted to allow the user to search for transaction records within the system, wherein a transaction record is a record of an activity that occurs in the system; and
an adjustment module adapted to allow the user to edit/revise transaction records.

8. A system for pharmaceutical samples management, the system comprising:
means for creating allocation models that specify quantities of samples that are allocated for specified time periods;
means for attaching schedules to the allocation models to specify time periods for providing feedback, ordering samples and receiving shipments of samples;
means for providing feedback regarding quantities specified in allocation models;
means for ordering samples allocated in allocation models;
means for tracking shipments of ordered samples;
means for providing inventory data regarding samples; and
means for analyzing data regarding samples programs.

9. The system of claim 8, further comprising a database that includes:
an operational database for storing data for transaction processing data;
a data warehouse database for storing data extracted from the operational database in a form suitable for use by the reports module and the analytics module;
a system control database for storing system configuration elements, such as database connectivity, system folder structures and files names, web server configuration details; and
an import/export database for storing data imported from and/or exported to external sources and the samples management system.

10. The system of claim 8, further comprising a means for specifying allocation schedules, order schedules, shipping schedules and inventory schedules.

11. The system of claim 8, wherein the means for crating allocation models further comprises means for displaying, providing feedback, editing and approving quantities of samples specified in allocation models.

12. The system of claim 8, wherein the means for creating allocation models further comprises:
means for importing externally-generated allocation models;
means for creating allocation models based on historical samples data for each sales team;
means for creating allocation models based on scripts data for a specific prescriber; and
means for creating an allocation model based on a specified quantity of particular samples, for particular sale teams, for particular time periods;
means for displaying, editing and/or approving an existing allocation model;
means for providing feedback regarding an allocation model; and
means for controlling how samples are made available to a sales team.

13. The system of claim 8, wherein the means for creating allocation models further comprises:
means for importing an externally-generated order;
means for creating orders based on pre-defined templates;
means for creating an order for one or more samples for a single user;
means for creating an order for one or more samples for multiple users;
means for displaying details about orders, such as tracking details, details about individual orders, lists of multiple orders; and
means for automatically generating an order when an inventory level for a specified sample falls to a specified threshold.

14. The system of claim 8, wherein the means for creating allocation models further comprises:
means for searching for transaction records within the system, wherein a transaction record is a record of an activity that occurs in the system; and
means for editing/revising transaction records.

15. A computer program product for implementing a pharmaceutical samples management system, the computer program product comprising:
a machine readable storage medium;
machine code stored in the machine readable storage medium which when executed by a computer processor configures the computer processor to:
allow a user to create allocation models for pharmaceutical samples, wherein an allocation model specifies quantities of pharmaceutical samples that are allocated amongst a sales team for specified time periods;
allow the user to order pharmaceutical samples based on an allocation model;
allow the user to monitor a system transaction;
allow the user to perform samples inventory reconciliation;
allow the user to obtain reports regarding the system;
allow the user to obtain analytical information regarding the system;
allow the user to display data regarding the system; and
provide alert information regarding the system.

16. The computer program product of claim 15, wherein the machine code further configures the computer processor to:
store data for transaction processing data in an operational database;
store data extracted from the operational database in a data warehouse database in a form suitable for providing report and analytical information;
store system configuration elements, such as database connectivity, system folder structures and files names, web server configuration details in a system control database; and
store data imported from and/or exported to external sources in an import/export database.

17. The computer program product of claim 15, wherein the machine code further configures the computer processor to allow the user to specify allocation schedules, order schedules, shipping schedules and inventory schedules.

18. The computer program product of claim 15, wherein the machine code further configures the computer processor to allow the user to display, provide feedback, edit and approve quantities of samples specified in allocation models.

19. The computer program product of claim 15, wherein the machine code further configures the computer processor to allow the user to:
import externally-generated allocation models;
create allocation models based on historical samples data for each sales team;
create allocation models based on scripts data for a specific prescriber; and
create an allocation model based on a specified quantity of particular samples, for particular sale teams, for particular time periods;
display, edit and/or approve an existing allocation model;
provide feedback regarding an allocation model; and
control how samples are made available to the sales team.

20. The computer program product of claim 15, wherein the machine code further configures the computer processor to allow the user to:
import an externally-generated order.
create orders based on pre-defined templates;
create an order for one or more samples for a single user;
create an order for one or more samples for multiple users;
display details about orders, such as tracking details, details about individual orders, lists of multiple orders; and
specify that the system should automatically generate an order for the user when an inventory level for a specified sample falls to a specified threshold.

21. The computer program product of claim 15, wherein the machine code further configures the computer processor to allow the user to:
allow the user to search for transaction records within the system, wherein a transaction record is a record of an activity that occurs in the system; and
edit/revise transaction records.
